(19) United States
(12) Patent Application Publication
(10) Pub. No.: US 2022/0134338 A1
(43) Pub. Date: May 5, 2022

(54) DIAGNOSTIC ASSAY SYSTEM WITH REPLACEABLE PROCESSING MODULES AND REMOTE MONITORING

(71) Applicant: Cepheid, Sunnyvale, CA (US)

(72) Inventors: Ronald Chang, Redwood City, CA (US); Steven M. Montgomery, Sunnyvale, CA (US); Gregory E. Mote, Sunnyvale, CA (US); Brian M. Bliven, San Jose, CA (US)

(21) Appl. No.: 17/516,084

(22) Filed: Nov. 1, 2021

Related U.S. Application Data
(60) Provisional application No. 63/107,934, filed on Oct. 30, 2020.

(51) Int. Cl. B01L 3/00

(52) U.S. Cl. CPC B01L 3/502715; B01L 3/502738; B01L 2200/0689; B01L 2200/16; B01L 2400/06; B01L 2200/145; B01L 2300/087; B01L 2400/0478; B01L 2200/04

(57) ABSTRACT

A biological sample processing apparatus having an enclosure and a plurality of sample processing modules held within an enclosure with a tiltable graphical user interface screen. In one aspect, the individual modules that are readily removable for repair, replacement or upgrade. Each module is configured to be independently operable and readily inserted into the enclosure for connection with a processing unit of the enclosure. Each module can include quick-release mechanisms so that the module can be readily removed and replaced manually or with minimal tools through the front of the enclosure without requiring substantial or total disassembly of the module or entire enclosure. In another aspect, the user interface screen can display identifying information, such as a barcode, that can be scanned by a user's portable device so as to monitor the progress of an assay remotely.

This application is a Non-Provisional of and claims the benefit of priority of Provisional Application No. 63/107,934 filed Oct. 30, 2020, which is incorporated herein by reference in its entirety.

This application is generally related to U.S. Pat. No. 10,132,728 entitled “Apparatus with Heterogeneous Processing Modules,” issued on Nov. 20, 2018; U.S. Pat. No. 6,660,228, entitled “Apparatus for Performing Heat-Exchanging, Chemical Reactions,” issued Dec. 9, 2003; U.S. Pat. No. 6,391,541, entitled “Apparatus for Analyzing a Fluid Sample”, issued May 21, 2002; Int'l Pub. No. WO/2000/072970, entitled “Cartridge for Conducting a Chemical Reaction,” and Int'l Pub. No. WO/2000/073412, entitled “Apparatus and Method for Analyzing a Fluid Sample,” the entire contents of which are incorporated herein by reference for all purposes.

### BACKGROUND OF THE INVENTION

The analysis of samples such as clinical or environmental samples generally involves a series of processing steps, which may include separate chemical, optical, electrical, mechanical, thermal, or acoustical processing of the samples. Many conventional diagnostic assay systems shuttle a sample cartridge or container between various different processing locations at which various steps of sample processing and testing are performed. In some diagnostic apparatuses, such as the GeneXpert by Cepheid, a sample cartridge is processed while the sample remains within a sample cartridge or attached reaction vessel. In the GeneXpert apparatus, the sample cartridge is inserted within a sample processing module that performs the various sample processing steps, typically from sample preparation to analytical testing, after which the spent sample cartridge is removed from the module. Thus, the sample is processed and tested while the sample cartridge remains at a single location within the module. In order to increase sample throughput, such apparatus often include multiple such modules disposed within a common enclosure. The enclosure is equipped with an internal computer and power sources to power and communicate with the individual modules.

Due to the burden and wear-and-tear of sample processing, one or more modules may periodically require maintenance and replacing. Further, over time, components of the module may be replaced or become obsolete and require development of new modules. Accordingly, currently replacing modules requires disassembling the apparatus and individual modules to some extent, which can be time-consuming and require significant down time. Therefore, there is a need for an apparatus that facilitates periodic ready removal, repair and replacement of repaired or updated modules with improved ease of use. Further, performing multiple concurrent assays can be cumbersome and time-consuming such that there is need for improved configurations that improve ease of use.

### BRIEF SUMMARY OF THE INVENTION

Some embodiments of the invention relate to a biological sample processing apparatus having an enclosure with multiple processing modules therein.

In one aspect, the invention pertains to an apparatus having readily removable and replaceable processing modules. In some embodiments, the modules can be readily removed from the enclosure manually or with a single tool without requiring disassembly of the module or the enclosure. In some embodiments, replacement entails opening or removing one or more front access panels, while the enclosure itself and internal components therein remain intact. In some embodiments, the front access panel(s) can be removed by removal of less than four screws, typically, a single screw. In some embodiments, the invention pertains to a replacement module having new or improved components that is configured to be backwards-compatible for drop-in replacement in an existing apparatus, such as the GeneXpert systems. In another aspect, the invention pertains to modules configured so that one or more components are readily removable and replacement to allow replacement and improvement with next-generation components as they become available and older components become obsolete.

In some embodiments, the apparatus and modules are configured such that one or more modules, components or component assemblies are readily removable to facilitate repair, update and replacement as needed. In some embodiments, the apparatus components include any or all of: a CPU, including a communication unit and processing unit, and power supplies for individual modules within the enclosure. In some embodiments, the module components include any or all of: a valve drive, a syringe drive, a sonication horn, an instrument assembly, including a thermal cycling unit, or any combination thereof. In some embodiments, any of the above components can be configured to be readily removed and replaced to facilitate repair, update and replacement of the components as needed. In some embodiments, one or more of the components are constructed in a modular manner such that the components can be readily removed without requiring substantial or complete disassembly of the entire apparatus or module. In some embodiments, “without disassembly” means that front access panels can be removed, without disassembly of the enclosure and associated internal components. In some embodiments, the front access panel can be removed upon removal of less than four screws, two or less screws, typically by removal of a single screw. This approach inhibits removal of an unauthorized user without tools, while still allowing for ready removal and replacement of one or more modules.

In another aspect, the module includes additional components and/or functionality to facilitate sample processing that include any or all of: CPU connectivity, module servicing, system sample cartridge identification, a door mechanism for loading/unloading of the sample cartridge and thermocycling units. Sample cartridge identification can include barcode scanners, cameras, NFID or RFID detection, or any suitable identification means. In some embodiments, the module includes one or more features to facilitate and improve CPU connectivity. In some embodiments, the module includes one or more features to facilitate module servicing, including slidable tracks for inserting the module and one or more quick-release mechanisms to allow for ready removal of the module. In some embodiments, the module includes one or more features to facilitate and improve upon the module's thermal cycling ability, such as the use of gradient cooling by independently controlled heaters, or active cooling by a Peltier device. In some embodiments, the module includes one or more features to facilitate and improve upon cooling of the entire apparatus. The cooling features can include a directed airstream concept, including a filtered airstream, a closed/sealed system with non-PCR heating sources removed from the enclosure, and/or thermal conduction by a heat sink, which can include the enclosure housing. In some embodiments, the module includes one or more features to facilitate and improve upon identification of the sample cartridges, such as a barcode scanner within the cartridge receiving bay of the module. In some embodiments, the apparatus includes a central identifier to scan a sample, a cartridge or a user badge. In some embodiments, the system includes a barcode reader within each individual cartridge bay configured to read an inserted sample cartridge and a centralized external barcode reader that is configured to read a user badge, cartridge or sample such that a given user can use either scanner for a given sample cartridge. In some embodiments, the module includes one or more features to facilitate and improve upon the door mechanism for loading/unloading of the sample cartridges.

In another aspect, the module includes one or more features to provide backwards compatibility with earlier apparatuses to allow a user to use new module within an earlier generation apparatus. In another aspect, the modules are configured for forward compatibility with next generation apparatus utilizing next generation modules.

In one aspect, the invention pertains to a plurality of sample processing modules held by the enclosure. Each sample processing module is configured to hold a removable sample cartridge and to only perform sample processing on a sample within the corresponding removable sample cartridge. Each sample processing module can be configured to perform at least one of a plurality of testing processes on the sample within the removable sample cartridge and to perform nucleic acid amplification and detection. Typically, the respective modules are configured to perform sample preparation, nucleic acid amplification and detection. In some embodiments, at least one sample processing module can be configured for hybridizing a nucleic acid to an array on a solid support. In some embodiments at least one sample processing module can be configured for nucleic acid amplification and detection in a multiplex array of wells, wherein each separate well comprises a separate nucleic acid amplification reaction. In some embodiments, each of the separate wells of the multiplex array of wells is capable of carrying out a multiplex reaction (e.g. nested PCR). In some embodiments, at least one sample preparation module can be configured to prepare a sample to undergo a sample processing protocol for at least one nucleic acid. In some embodiments, at least one sample processing module can be configured for detection of at least one protein analyte. In some embodiments, at least one sample processing module can be configured to perform immunoassays. In some embodiments, at least one sample processing module can be configured for assessing a chromosomal copy number of at least one gene of interest. In some embodiments, at least one sample processing module can be configured for performing a multiplex detection of at least two nucleic acid analytes. In some embodiments, at least one sample processing module can be configured for performing a multiplex detection of at least two protein analytes. In some embodiments, at least one sample processing module can be configured for sequencing and detecting a nucleic acid molecule. In some embodiments, the plurality of sample processing modules can include at least one module for detecting at least one protein analyte contained within a biological sample within a test cartridge, at least one module for assessing chromosomal copy number of at least one gene of interest contained within a biological sample within a test cartridge; and at least one module for performing a sample processing protocol for at least one nucleic acid contained within a biological sample within a test cartridge. In some embodiments, the plurality of sample processing modules includes different modules configured for different types of sample processing.

In some embodiments, the plurality of sample processing modules can include at least one module that can be configured for hybridizing a nucleic acid to an array on a solid support and/or at least one module that can be configured for detection of at least one protein analyte and/or at least one module that can be configured for assessing a chromosomal copy number of at least one gene of interest and/or at least one module that can be configured for performing a multiplex detection of at least two nucleic acid analytes and/or at least one module that can be configured for performing a multiplex detection of at least two nucleic acid analytes and/or at least one module that can be configured for performing a multiplex detection of at least two protein analytes and/or at least one module that can be configured for sequencing and detecting a nucleic acid molecule and/or at least one module that can be configured for performing PCR and/or at least one sample processing module that can be configured for performing rapid PCR.

In some embodiments, the plurality of sample processing modules can be up to 16 sample processing modules made up of a combination of modules, which in some embodiments are different types of modules, such as 1, 2, 3, 4, 5, 6, 7, 8, 9, 10, 11, 12, 13, 14, or 15 modules (depending on whether other types of modules are included within the plurality). The modules all be of the same type and functionality or can be of differing types, including differing functionality, differing construction and same or similar functionality.

Some embodiments of the invention relate to a method for operating a sample processing apparatus that includes a housing having multiple processing modules therein. In the method, a sample cartridge holding an unprepared sample at one of a plurality of sample processing modules held by an enclosure can be received. As the cartridge is received, an identifier, such as a barcode scanner identifies the sample and sample cartridge. The user can interface with the apparatus by a central display on the enclosure housing and monitor status of the sample processing by the central display. The central display is configured to selectively display information of multiple parameters of the assay being performed, which can include identifying information of a module, a sample cartridge, a patient, an assay, or status information as to a particular assay being performed. The central display is configured to display information as to any of the one or more modules therein upon receiving a selection by a user via the touch display or automatically as needed (e.g. opening of the door, an error occurring, completion of the assay). While the sample is processing, another sample cartridge can be received within another module in the same manner, while the user interfaces with the apparatus via the central display.

Some embodiments of the invention relate to methods of removing and replacing modules within a sample processing apparatus that includes an enclosure housing having multiple processing modules therein. In the method, the user may tilt up a central display, remove any front access panels attached to the front of the enclosure, release one or more quick-release mechanisms and slidably remove one or more modules from the enclosure. A repaired or updated module can be replaced and inserted within the enclosure by sliding the module along one or more tracks and slidably connecting rear facing connectors on the module to plug-in type connectors in the rear of the enclosure.

In another aspect, the system can be configured such that the graphical user interface screen display selectable options that enable the user to remotely access a status, completion or result of the assay, or displays a unique on-screen identifier corresponding to a particular assay being run that, when scanned, enables the user to remotely access a website displaying the status, completion or result of the assay. In some embodiments, the selectable option communicates with a communication unit (e.g. by SMS) a link to the user's device to a website displaying information from a task record of the assay. The communication unit can utilize any of wired or wireless connections (e.g. NFC, Wifi, cellular) or any combination thereof. In some embodiments, the identifier is a URL-linked QR code found on the molecular diagnostic device's screen. The QR code can be scanned by a user's QR-recognizing mobile device, to enable the user to access, via the internet, real-time information from the task record of the assay being performed, thus creating walk-away capabilities to users so that the user can monitor a status and/or result of the assay remotely. In some embodiments, the test status/results may be linked to multiple mobile devices, such as smartphones or tablets. In an exemplary embodiment, the system generates and displays a unique QR code associated with a particular assay, the user captures an image of the QR code with the camera of their mobile device (e.g. smartphone), which allows the user to access a URL that shows the user a status of the test being run—remotely. In other embodiments, the display allows the user to select the option of texting (SMS) or emailing the user a link to the URL to one or more users by which the user(s) can also monitor the assay.

### BRIEF DESCRIPTION OF THE DRAWINGS

FIGS. 1A-1B show a biological sample processing apparatus and biological sample cartridges in accordance with some embodiments of the invention.

FIGS. 2A-2C show a biological sample processing apparatus in accordance with embodiments of the invention.

FIG. 3 shows conventional biological sample processing apparatus and associated peripherals.

FIG. 4 shows a control unit module to replace conventional peripherals of conventional apparatus, in accordance with some embodiments.

FIGS. 5A-5B show a control unit module to replace conventional peripherals of conventional apparatus, in accordance with some embodiments.

FIG. 6 shows compatibility of the control unit module for interfacing with earlier generation apparatus and next generation apparatus, in accordance with some embodiments.

FIG. 7 shows manual loading of a sample cartridge into a sample processing apparatus, in accordance with some embodiments.

FIGS. 8A-8E show specialized removal tools to facilitate removal of a sample processing module from the apparatus enclosure, in accordance with some embodiments.

FIGS. 9-11 shows removal of a processing module from the apparatus enclosure, in accordance with some embodiments.

FIG. 12 shows a processing module removed from the apparatus enclosure, in accordance with some embodiments.

FIG. 13 shows another processing module being removed from the apparatus enclosure, in accordance with some embodiments.

FIGS. 14-15 shows a processing module configured with directed air cooling, in accordance with some embodiments.

FIGS. 16-17 shows an apparatus configured for directed air cooling and filtering, in accordance with some embodiments.

FIG. 18 shows a sealed, closed system, in accordance with some embodiments.

FIG. 19 shows a sample cartridge being loaded into a sample processing module and being identified by the module, in accordance with some embodiments.

FIG. 20 shows a bay door of a sample processing module, in accordance with some embodiments.

FIG. 21 shows a thermal cycling module of a sample processing module, in accordance with some embodiments.

FIGS. 22A-221 show differing sample processing modules depicting varying compatibility between previous and next generation apparatus in accordance with some embodiments.

FIGS. 23-34 shows a sample processing apparatus configured for being powered by a direct connection or by a battery pack for portability, in accordance with some embodiments.

FIG. 25 shows a portable battery pack, in accordance with some embodiments.

FIG. 26 shows an apparatus with a removable CPU module, in accordance with some embodiments.

FIG. 27 shows a sample processing apparatus having a display output for an external monitor, in accordance with some embodiments.

FIG. 28 shows a schematic of the communication scheme facilitating remote monitoring of the assay by a user, in accordance with some embodiments.

FIG. 29 shows a schematic that illustrates generating a secure link for displaying information from a task record of an assay being remotely monitored, in accordance with some embodiments.

FIG. 30 shows a user setting facilitating communication of a link for remote monitoring of an assay, in accordance with some embodiments.

FIGS. 31A-31B show a user device displaying a basic data set for a task record and FIGS. 32A-32B shows a user device displaying an advanced data set for a task record in remote monitoring of an assay, in accordance with some embodiments.

FIG. 33 shows a user device displaying multiple tabs of information from task records of multiple assays being performed concurrently, in accordance with some embodiments.

FIG. 34 shows a flowchart of a sequence of operations in remote monitoring of an assay test while running, in accordance with some embodiments.

FIG. 35 shows a flowchart of a sequence of operations in remote monitoring of an assay test result, in accordance with some embodiments.

### DETAILED DESCRIPTION OF THE INVENTION

Embodiments of the invention pertain to an apparatus for performing multiple types of assays and related sample preparation. The apparatus can include multiple sample processing modules, typically having, or capable of having up to 15 different types of modules. The modules can be configured for different types of assays (e.g., immunoassay, PCR, rapid PCR, sequencing, chromosomal analysis, and flow cytometry, etc.) for detecting different types of target analytes (e.g., nucleic acid, whole cell, DNA, RNA, protein, virus, drugs, etc.). The apparatus can also include modules dedicated to sample preparation (e.g., lysis, chemical treatment, filtration, etc.). A cartridge-based sample holder is standardized for each type of module, so that in most cases each module can interface with the same cartridge. The modules, regardless of type, can all share the same chassis footprint and electronic interface, such that types of modules can be changed with little difficulty. The enclosure and modules are configured to allow for ready removal and replacement with updated modules or componentry.

As used herein, the term “biological sample” (interchangeable with “test sample” or “sample”) encompasses any material that may contain an analyte of interest (e.g., a particular protein or nucleic acid), often taken from or otherwise derived from a living organism. “Biological samples” may include, but are not limited to, samples of tissues such as biopsy and autopsy samples, and frozen or paraffin embedded sections taken for histological or pathology purposes. Such samples may include whole blood, serum, plasma, cerebrospinal fluid, sputum, tissue, cultured cells, e.g., primary cultures, explants, transformed cells, stool, urine, vesicle fluid, mucus, and other bodily secretion, or tissue that could be sampled with a swab device. Furthermore, in some cases, a “biological sample” can be material taken from an environment (e.g., water, air, soil, and the like) where the presence of a particular organism may be suspected.

As used herein, the term “configured” describes a particular arrangement of hardware components, such as chassis, heaters, fans, optical sensors, fluid couplings, fluid passages, microfluidics, piezoelectric components, processor, memory containing instructions, supporting circuitry, and/or connectors, etc.

As used herein, the term “sample processing module” (interchangeable with “processing module” and “module”) is defined as a modular sub-portion of the testing system, which has a particular physical form factor compatible with the system and includes hardware components (heaters, fans, optical sensors, fluid couplings, fluid passages, microfluidics, piezoelectric components, processor, memory containing instructions, supporting circuitry, and/or connectors, etc.) configured to perform a particular process for a sample, which can include any or all of a sample preparation and analytical testing process.

As used herein, the term “sample preparation” is defined as a process typically performed prior to one or more particular assays. The process changes a physical characteristic of a sample prior to the assay(s), for example, by physical, chemical, and/or enzymatic treatment (e.g., lysis by sonification, enzymatic, detergents, solvents, cell-bomb, etc., filtration, and/or concentration).

As used herein, the term “assay” (interchangeable with “testing process” and “biological testing process”) is defined to be an investigative procedure performed on a sample, including but not limited to, determining the presence/absence and/or the quantity/concentration of a particular analyte.

Non-limiting exemplary analytes can include any nucleic acids and/or proteins, analytes specific for bacterial pathogens (e.g. methicillin resistant Staphylococcus aureus, C. difficile, tuberculosis, group B strep., chlamydia, and gonorrhea), viral pathogens (e.g. influenza, Covid-2, RSV, HIV, HCV, and HBV), tumor cells (e.g., bladder cancer, lung cancer, breast cancer, colon cancer, and leukemia), biothreat analytes such as anthrax or ricin, chromosomal alterations, such as gene duplication, gene deletions or gene translocations, cells expressing specific cell surface markers such as CD4+ cells, detection of gene mutation/alterations such as single nucleotide polymorphisms (SNPs) and methylation status of genes.

As used herein, the term “removable sample cartridge” (interchangeable with “sample cartridge” and “cartridge”) refers to a specialized container for holding a sample that is configured to temporarily physically interface with a sample processing module such that control aspects (fluid connections, heaters, piezoelectric components, optical sensors, etc.) of the sample processing module can directly or indirectly perform a process on the sample within the container, after which the removable sample cartridge can be removed from the sample processing module to further analyze, process, or dispose of the sample. The removable sample cartridge couples and uncouples with the sample processing module without the need for using additional tools (e.g., screwdriver, hex-key, etc.) to fasten the removable sample cartridge to the sample processing module, akin to an electrical plug interfacing with an electrical wall outlet, except for cases of jamming or other malfunction, which may require such tools to help remove the cartridge. In some embodiments, the removable sample cartridge may contain, or has physical aspects for receiving, particular chemicals, such as primers and reagents (including reactants).

In this application, the term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, mutations including point mutations, single nucleotide polymorphisms (SNPs), and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)). The term nucleic acid is used interchangeably with gene, cDNA, and mRNA encoded by a gene.

The term “gene” means the segment of DNA involved in producing a polypeptide chain; it includes regions preceding and following the coding region (leader and trailer) involved in the transcription/translation of the gene product and the regulation of the transcription/translation, as well as intervening sequences (introns) between individual coding segments (exons).

A “polynucleotide hybridization method” as used herein refers to a method for detecting the presence and/or quantity of a polynucleotide based on its ability to form Watson-Crick base-pairing, under appropriate hybridization conditions, with a polynucleotide probe of a known sequence.

In this application, the terms “polypeptide,” “peptide,” and “protein” are used interchangeably to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymers. As used herein, these terms encompass amino acid chains of any length, including full-length proteins (i.e., antigens), wherein the amino acid residues are linked by covalent peptide bonds. The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine.

As used herein, the terms “multiplex” and “array” refer to an assay format that permits simultaneous detection and/or quantification of multiple analytes (e.g., dozens or more of the same or different molecules) in a single run/cycle of the assay.

As used herein, the term “solid support” refers to an inert solid material, which may be a natural material, such as glass and collagen, or a synthetic material, such as acrylamide, cellulose, nitrocellulose, silicone rubber, polystyrene, polyethylene vinyl acetate, polypropylene, polymethacrylate, polyethylene, polysilicates, polyethylene oxide, polycarbonates, teflon, fluorocarbons, nylon, polyanhydrides, polyglycolic acid, polylactic acid, polyorthoesters, polypropylfumarate, glycosaminoglycans, and polyamino acids. One example is silica gel preimpregnated with fluorogenic substrates. A “solid support” typically provides a supporting structure for performing an assay in various apparatus of this application.

Replaceable Sample Processing Modules and System

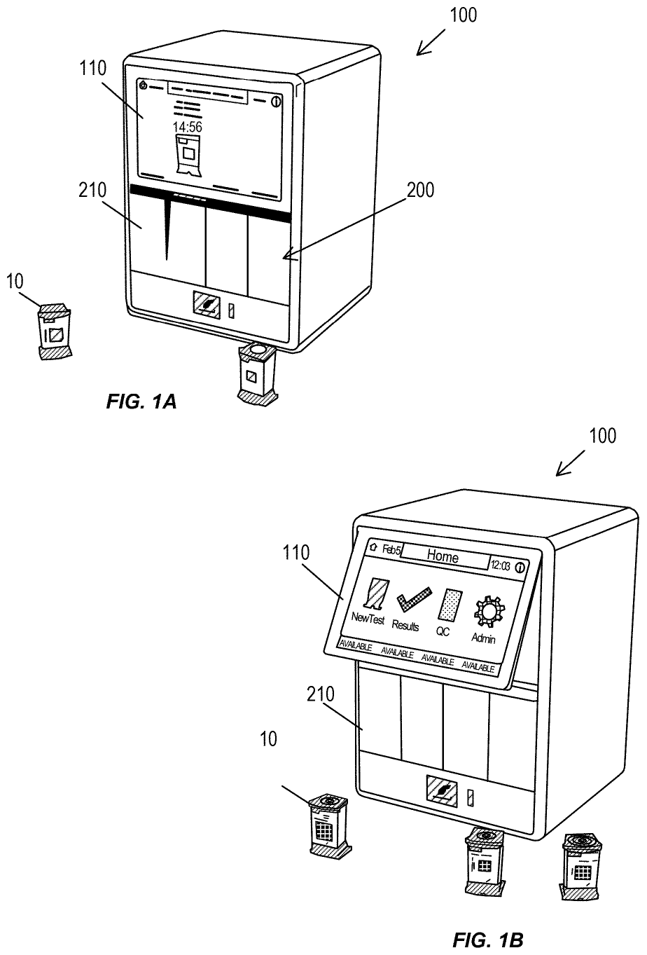

The apparatus and modules of the present invention can be further understood by referring to the detailed examples of FIGS. 1A-27 and the description provided below.

FIGS. 1A-1B shows a biological sample processing apparatus 100, according to some embodiments of the invention. The apparatus 100 includes multiple, typically four, processing modules 200 held within an enclosure. Each module includes a door 210 covering a bay for receiving a biological sample cartridge 10. The module population can be homogeneous in nature, where all the modules are identical, or can be heterogeneous in nature, such that the modules do not necessarily perform the same processing tasks. For example, processing modules can include PCR processing modules, array modules, and dedicated sample preparation modules (e.g., lysis by sonification, enzymatic, detergents, solvents, cell-bomb). In some embodiments, the modules can be functionally identical, but the module population encompasses differing version of the modules, such as an earlier generation of module and a next generation module having one or more updated components or sub-modules.

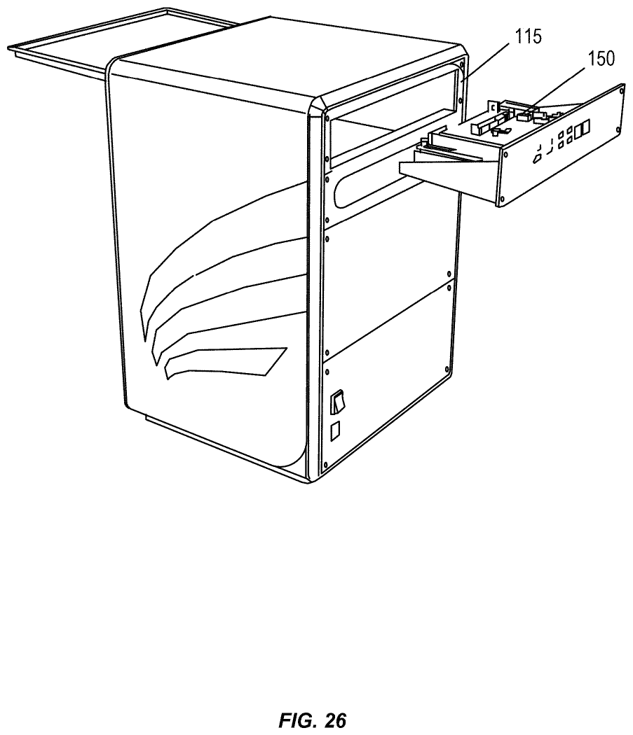

The sample processing modules are connected by a communications bus to a control unit having a controller (for example, see control unit module 150 in FIG. 26). The control unit is configured to independently operate each sample processing module 200. The control unit can be, for example, a general purpose or specific purpose computer. The control unit generally includes at least one processor and supporting circuitry, and memory storing instructions for independently operating each sample processing module. In some embodiments, the control unit is structurally integrated into the apparatus 100. The control unit may be readily removable for repair or upgrade. In other embodiments, the control unit is remotely connected to the apparatus via a wired or wireless connection, such that the control unit can be depicted as personal computer. The apparatus 100 has a main logic board with edge connectors for establishing electrical connections to the modules 200. The apparatus 100 also preferably includes a fan for cooling its electronic components. The apparatus 100 may be connected to the controller using any suitable data connection, such as a universal serial bus (USB), ethernet connection, or serial line, or the controller may be built into the apparatus 100.

Generally, each sample processing module 200 will share the same structural format and can be configured to electronically interface with the enclosure via a shared type of connector inside the enclosure. This arrangement allows for easy swapping of modules when different configuration needs arise for the user. Each sample processing modules 200 is configured to interface with a sample testing cartridge 10, for example, such as the vessel disclosed in FIG. 1 of commonly assigned U.S. Pat. No. 6,660,228, entitled “APPARATUS FOR PERFORMING HEAT-EXCHANGING, CHEMICAL REACTIONS, which is incorporated by reference, and also such as, for example, the vessel disclosed in FIG. 1 of commonly assigned U.S. Pat. No. 6,391,541, entitled “APPARATUS FOR ANALYZING A FLUID SAMPLE”, which is incorporated by reference herein. In some embodiments, the same cartridge can be used in any of the sample processing modules. Aspects of Int'l Pub. No. WO/2002/18902, entitled “FLUID METERING AND DISTRIBUTION SYSTEM”, Int'l Pub. No. WO/2000/072970, entitled “CARTRIDGE FOR CONDUCTING A CHEMICAL REACTION”, and Int'l Pub. No. WO/2000/073412, entitled “APPARATUS AND METHOD FOR ANALYZING A FLUID SAMPLE”, can also be used in any of the sample processing modules. These references are incorporated by reference herein.

In some embodiments, the sample processing module 200 is configured as a sample preparation module to prepare a sample for later processing (e.g., lysis by ultrasonification). An example of such a configuration is shown in commonly assigned U.S. Pat. No. 6,739,537, entitled “APPARATUS AND METHOD FOR RAPID DISRUPTION OF CELLS OR VIRUSES”, which is incorporated by reference. Another example of such a configuration is shown in commonly assigned U.S. Pub. No. US 2010/0129827, entitled “METHOD AND DEVICE FOR SAMPLE PREPARATION CONTROL”, which is incorporated by reference.

In some embodiments, flow cytometry is one of the detection methods that can be used in one or more sample processing modules for detecting the presence of a predetermined target, such as a certain cell type or a population of cells that express a particular marker. Methods and instrumentation for practicing flow cytometry are known in the art, and can be used in the practice of the present invention. Flow cytometry in general resides in the passage of a suspension of cells or microparticles comprising a label (e.g. a fluorophore) as a stream past a laser beam and the detection of the label (e.g. fluorescent emission) from each particle by a detector, such as a photo multiplier tube. Detailed descriptions of instrumentation and methods for flow cytometry are found in the literature. Examples are McHugh, “Flow Microsphere Immunoassay for the Quantitative and Simultaneous Detection of Multiple Soluble Analytes,” Methods in Cell Biology 42, Part B (Academic Press, 1994); McHugh et al., “Microsphere-Based Fluorescence Immunoassays Using Flow Cytometry Instrumentation,” Clinical Flow Cytometry, Bauer, K. D., et al., eds. (Baltimore, Md., USA: Williams and Williams, 1993), pp. 535-544; Lindmo et al., “Immunometric Assay Using Mixtures of Two Particle Types of Different Affinity,” J. Immunol. Meth. 126: 183-189 (1990); McHugh, “Flow Cytometry and the Application of Microsphere-Based Fluorescence Immunoassays,” Immunochemica 5: 116 (1991); Horan et al., “Fluid Phase Particle Fluorescence Analysis: Rheumatoid Factor Specificity Evaluated by Laser Flow Cytophotometry,” Immunoassays in the Clinical Laboratory, 185-189 (Liss 1979); Wilson et al., “A New Microsphere-Based Immunofluorescence Assay Using Flow Cytometry,” J. Immunol. Meth. 107: 225-230 (1988); Fulwyler et al., “Flow Microsphere Immunoassay for the Quantitative and Simultaneous Detection of Multiple Soluble Analytes,” Meth. Cell Biol. 33: 613-629 (1990); Coulter Electronics Inc., United Kingdom Patent No. 1,561,042 (published Feb. 13, 1980); and Steinkamp et al., Review of Scientific Instruments 44(9): 1301-1310 (1973). These references are incorporated herein by reference.

In some embodiments, one or more of the sample processing modules can be configured for detection of nucleic acids and/or proteins. Basic texts disclosing general methods and techniques for detection of nucleic acids and proteins include Sambrook and Russell, Molecular Cloning, A Laboratory Manual (3rd ed. 2001); Kriegler, Gene Transfer and Expression: A Laboratory Manual (1990); and Ausubel et al., eds., Current Protocols in Molecular Biology (1994). These references are incorporated herein by reference. A variety of polynucleotide amplification methods are well established and frequently used in research. For instance, the general methods of polymerase chain reaction (PCR) for polynucleotide sequence amplification are well known in the art and are thus not described in detail herein. For a review of PCR methods, protocols, and principles in designing primers, see, e.g., Innis, et al., PCR Protocols: A Guide to Methods and Applications, Academic Press, Inc. N.Y., 1990, which is incorporated by reference herein. PCR reagents and protocols are also available from various commercial vendors.

The apparatus 100 has a main logic board with edge connectors for establishing electrical connections to the modules. The apparatus 100 also preferably includes a fan for cooling its electronic components. The apparatus 100 may be connected to the controller using any suitable data connection, such as a universal serial bus (USB), ethernet connection, or serial line. It is presently preferred to use a USB that connects to the serial port of controller. Alternatively, the controller may be built into the apparatus 100.

The processing modules 200 are preferably independently controllable so that different chemical reactions and sample preparations can be run simultaneously in the apparatus 100. The apparatus 100 is modular so that each processing module 200 can be individually removed from the apparatus 100 for servicing, repair, replacement or upgrade. Typically, each module is readily removable, for example by a quick-release connection, to facilitate quick and easy removal of a module. This modularity reduces downtime since all the processing modules are not off line to repair one, and the instrument 100 can be upgraded and enlarged to add more modules as needed.

Apparatus 100 further include a central display 110 for displaying status indicators or instructions regarding any of the modules 200. In addition, the display can be configured to display metrics, status information or instructions for all modules or any combination of modules. For example, the display may indicate remaining times for all modules, or for a subset of modules that are currently processing. In another example, the display may show a user instructions regarding a sample processing protocol or to remove spent sample cartridges from one or more modules in which sample processing is completed. In some embodiments, the display is a touch screen that allows a user to interface with or control the modules. For example, the user may initiate a sample testing by inputting commands and information through the display. In some embodiments, the central display 110 is tiltable so as to be more easily viewed by the user at multiple angles. As shown in FIG. 1A, the display can be used in an upright position, or as shown in FIG. 1B, the display can be tilted upwards. This is advantageous as the display can be adjusted based on the relative position of the user to facilitate ease of operation.

The apparatus 100 can also include a microprocessor or microcontroller containing firmware for controlling the operation of the apparatus and modules. The microcontroller communicates through a network interface 132 to the controller computer via, for example, a USB connector. In some embodiments, the apparatus 100 includes network interface inlet and outlet ports for receiving a data connection through inlet port and outputting data to another apparatus through outlet port. In other embodiments, the apparatus can be configured to send and receive data wirelessly by any suitable means. The apparatus 100 also includes a microprocessor or microcontroller containing firmware for controlling the operation of the apparatus 110 and modules 200. The microcontroller communicates through a network interface to the controller computer via, for example, a USB connector.

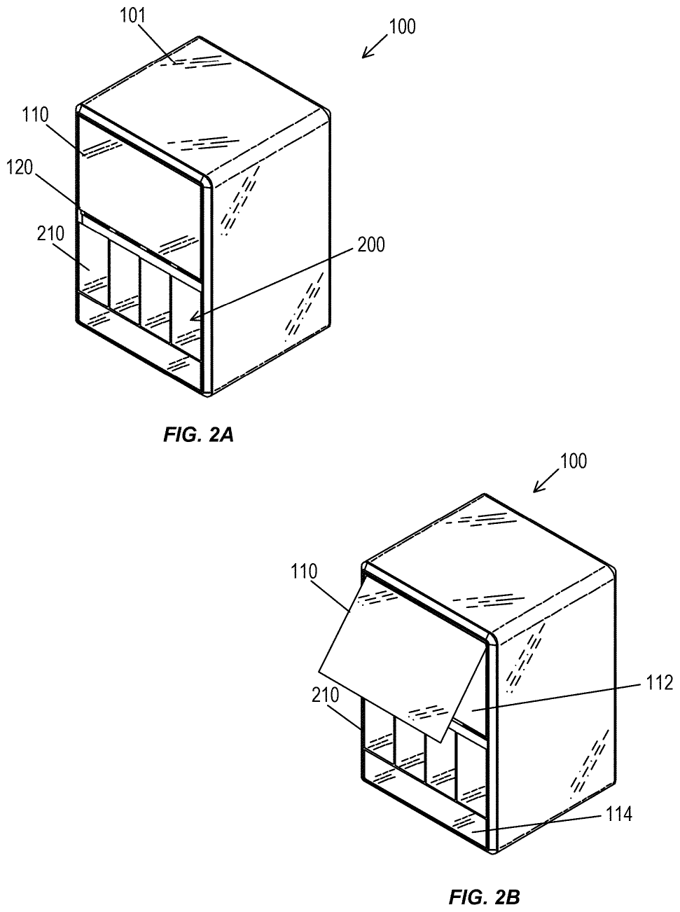

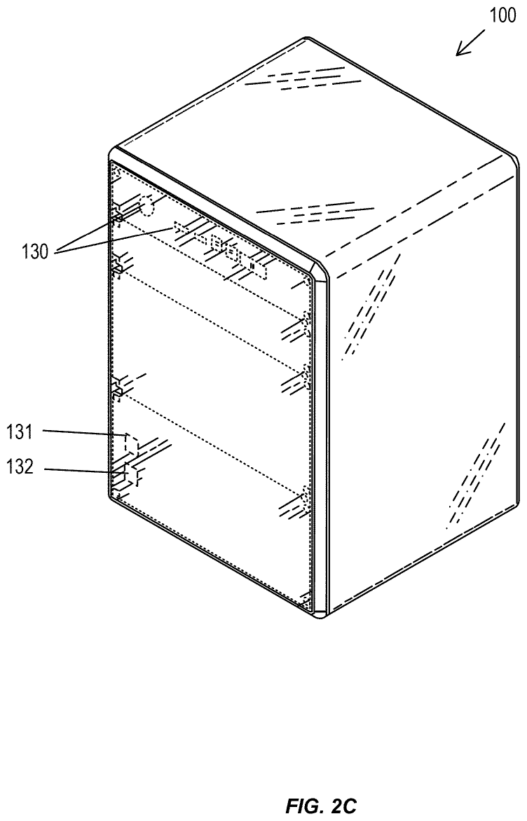

FIGS. 2A-2C show alternative views of a biological sample processing apparatus 100. The enclosure is defined by an outer housing or shell 101 that defines an interior cavity in which the multiple modules 200 are disposed. The housing or shell is typically made of a durable, rigid material, typically metal (e.g. aluminum) or any suitable material to prevent damage when transporting the apparatus. The modules 200 are inserted through a front opening of the housing and encased within the enclosure by removable front panels that attach on the front of the enclosure such that only the front bay doors 210 of the modules are exposed in the assembled apparatus. The front opening is covered by removable access panels, upper panel 112 and lower panel 114, that cover the upper and lower portions of the modules. By removal of the front access panels attached to the front of the enclosure, the modules can be readily removed without requiring disassembly of the enclosure itself. The tiltable screen 110 folds down over the upper panel 112. The upper panel 112 includes a lower status indicator portion that remains visible between the display 110 and the bay doors 210 (as shown in FIG. 2A). The status indicator portion includes multiple indicators 120 (e.g. LED lights) that correspond to each of the modules and that indicate a status of the module. For example, a solid light may indicate that module processing is complete, while a blinking light may indicate that the module is processing. The indicator portion of upper panel 112 includes corresponding connectors on a back portion thereof that releasably connect with a connector on the front-facing side of the module such that placing the upper panel 112 against the multiple modules connects the indicators on upper panel 112 to the connectors on the modules. The upper panel 112 and lower panel 114 can be readily removed manually, such as by use of snap-fit connectors, to allow the user to remove the panels to facilitate removal and replacement of the modules as needed (as shown in FIGS. 9-11). As can be seen in FIG. 2C, the rear side of the enclosure housing 101 includes panels with connectors for communication, external components and/or power. The upper portion can include multiple connectors 130 for communication inputs and outputs, while the lower portion includes power connector, such as power port 131 for a hard-wired power connection, and a power button 132. While the above demonstrates a particular configuration, it is appreciated that the noted connectors and panels could be devised according to various other configurations and locations and remain in keeping with the concepts described herein.

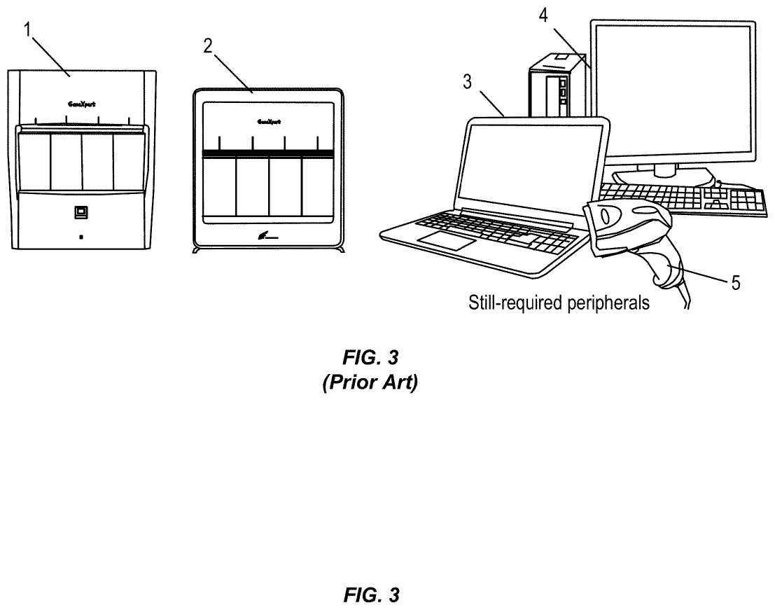

FIG. 3 depicts prior art biological processing apparatus having multiple processing modules for processing of biological sample cartridges, such as conventional apparatus 1 (GeneXpert R1) and conventional apparatus 2 (GeneXpert R2). These apparatuses rely on external peripherals to facilitate control of the processing modules as well as intake of the sample cartridges before processing in the apparatus. For example, in a typical process with these apparatuses, the user scans a biological sample cartridge with a cartridge identifier, such as barcode scanner 5, and initiates a sample processing procedure with an external computer, such as laptop 3 or desktop computer 4, that is communicatively coupled with one of these conventional apparatuses. While this approach represented a marked advancement when these apparatuses were introduced, this approach can be cumbersome, tedious and prone to human error, particularly when processing a high number of samples. In addition, the control software for the apparatus may be limited by the capabilities and availability of the external computer, which can be an issue in certain locations where use of the apparatus is urgently needed (e.g. remote locations where infections outbreaks may occur). Therefore, it would be desirable to devise an improved apparatus that provides the same or similar functionality without requiring the use of these additional peripherals or external computers. In addition, integrating the control unit, cartridge identifier and user interface within a single command input device, or even further integrating these devices into the apparatus itself, not only improves ease of use and renders use of external peripherals obsolete, but this allows for improved control and communications functionality beyond that allowed by available external peripheral devices.

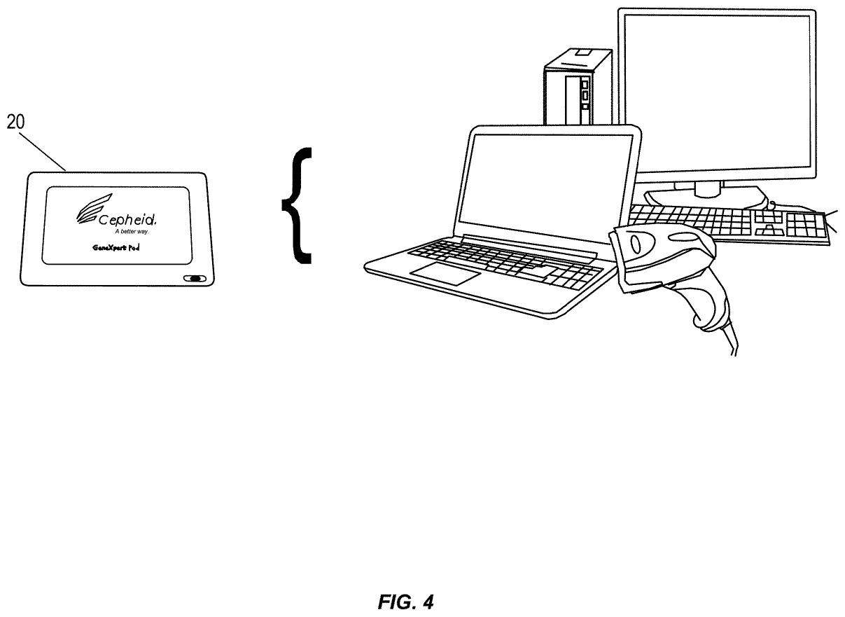

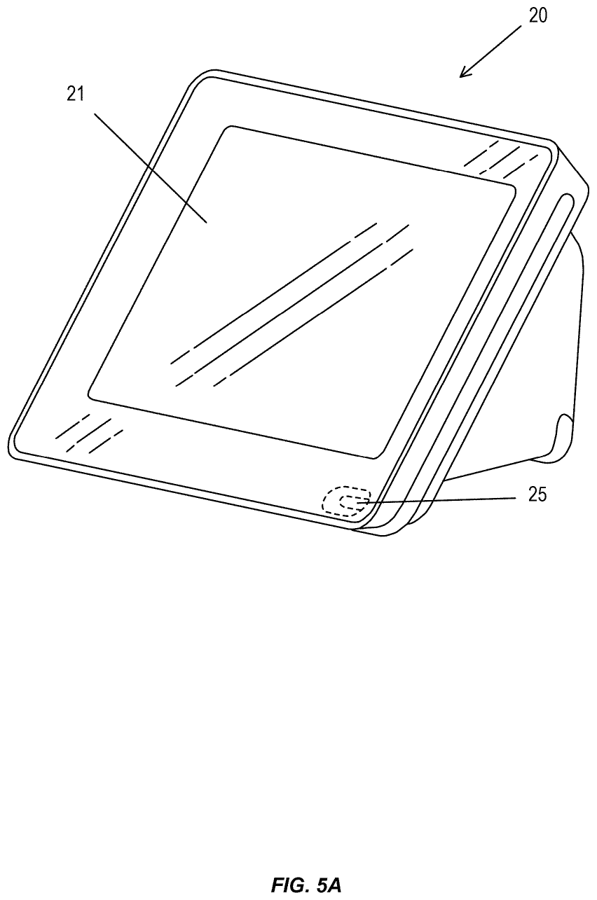

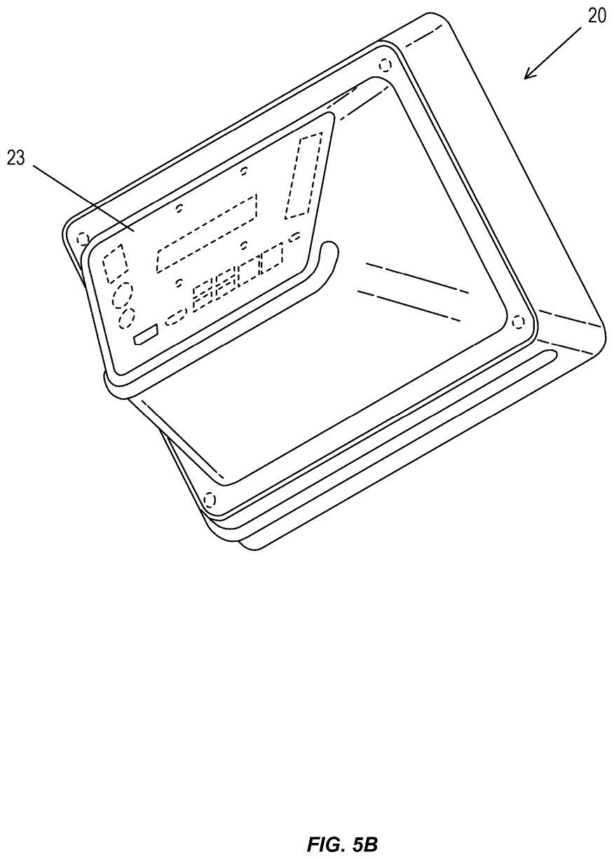

FIG. 4 depicts a single command input module 20 with an integrated control unit, user interface screen and cartridge identifier. This command input module is a specialized computer module with a processor and memory having software instructions recorded thereon for interfacing with and controlling the multiple modules of a biological sample processing apparatus that is communicatively coupled to the command input module 20. This approach is advantageous as it avoids reliance on available computer systems (e.g. laptop, desktop computer), which may have competing resources and limited functionality and may frequently be removed or switched out since such devices are general purpose computers. By integrating these functions into a specialized command input module that can only be used with the biological processing apparatus, this avoids competing with other resources and allows for more consistent availability, operation and capabilities.

As can be seen in FIGS. 5A-5B, command input module 20 includes a housing that includes a display screen 21, which is a touchpad to allow a user to enter information directly into the module. The module further includes a cartridge identifier 25, which can be configured as a barcode scanner, an RFID detector, or any suitable detection means. As shown in FIG. 5B, the rear side 23 of command input module 20 includes multiple connectors and/or receptacles for power and communications connections. The module communicatively connects to the biological processing apparatus by either hardwired connection or wirelessly. In some embodiments, the command input module can also be powered by a portable power source, such as a rechargeable battery. In some embodiments, the module can be connected to an external keyboard to facilitate data entry. In other embodiments, the module can wirelessly couple with an external computing device, such as a smartphone, laptop or desktop computer to facilitate data entry, or for uploading or downloading of sample data or sample testing data.

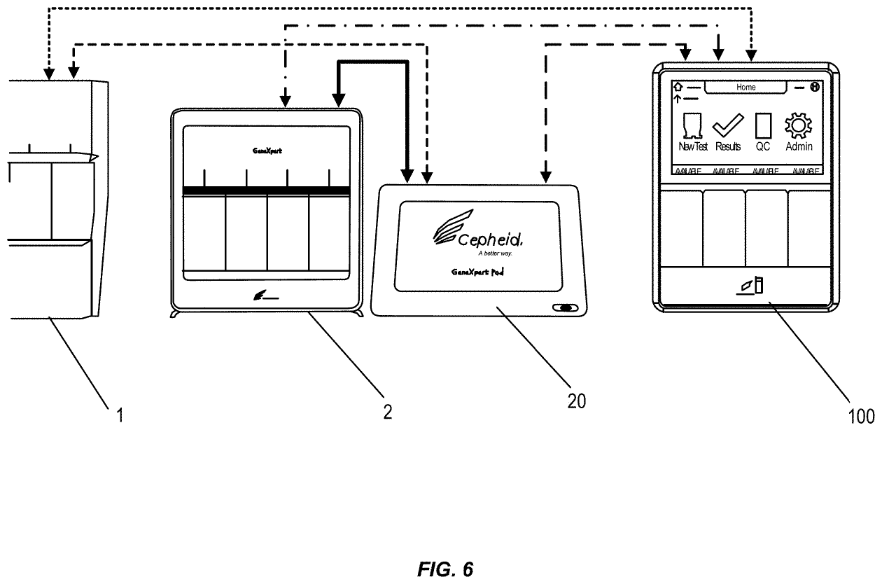

FIG. 6 shows that the command input module 20 is configured for use with conventional apparatus 1,2 so as to replace the external computer and peripherals previously needed for those apparatus. This aspect is advantageous for facilities that currently utilize one or more existing conventional apparatus. The use of command input module 20 allows the user to interface more efficiently and seamlessly with multiple apparatuses, rather than switching connections or laptops for each apparatus. In another aspect, the module can be used with the improved biological processing apparatus 100 described herein. This module can used to interface with and control apparatus 100 instead of the integrated control unit and display of the apparatus 100. This aspect may be desirable for users that already utilize the command input module 20 to control one or more conventional apparatuses, as it allows the user to control both conventional earlier generation apparatus and the next generation apparatus with the same command module. In some embodiments, the user can select a setting in apparatus 100 to effect control by the command input module 20. In some embodiments, the apparatus can automatically detect control being initiated by the command input so as to defer to the command input module, rather than the integrated control unit. Where control is effected through the command input module, the display 110 of the improved apparatus 100 may operate in a complementary fashion, for example, displaying the same display that appears on the command input module to allow a user to switch seamlessly between the displays. In another aspect, the display screen of the apparatus 100 may display other information, such as status information of all modules, while the command input module is being used to interface with one or more modules.

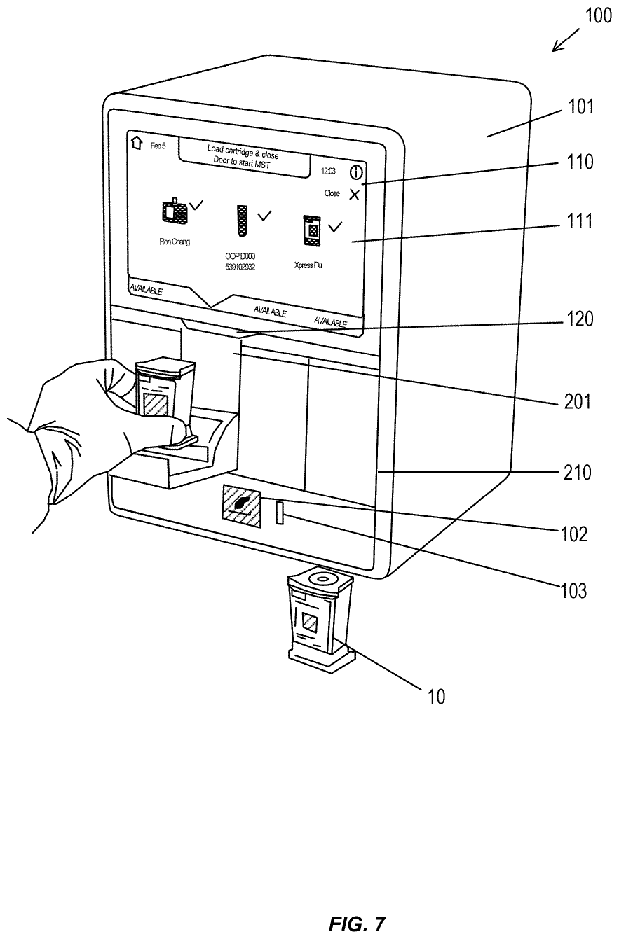

FIG. 7 shows manual loading of a sample cartridge into a sample processing apparatus 100, in accordance with some embodiments. The apparatus includes an enclosure housing 101 that holds multiple modules 200 configured for processing of biological sample cartridges received therein, as described previously. As shown, the user has initiated a sample processing procedure and is loading a sample cartridge 10 into a receiving bay of a module 200, the door 210 being in an open position and the display 110 displaying attributes of the sample procedure to be performed for the given module. The display is a touchpad 111 that allows the user to enter information and selection directly with the display. The indicator 120 light indicates the module 200 currently selected. An identifier 201 (e.g. barcode scanner) disposed inside the bay can read each cartridge and/or sample ID during loading. In some embodiments, the scanner can also detect a user badge of the personnel loading the device. The apparatus can also include a lower identifier 103 (e.g. barcode scanner) that can be used to identify a sample cartridge, sample or user badge. This apparatus further includes a soft on/off button 102 that can be styled as a logo. By utilizing an identifier to further identify a user, the apparatus can accommodate multiple, concurrent users. In some embodiments, the apparatus can include the same integral computer module as the command input module previously discussed. The apparatus can further be configured with a new visual language especially developed for the product line.

In another aspect, the apparatus is configured to allow for easy removal of the individual modules for repair or replacement. Whereas previous apparatus required some level of disassembly of the apparatus and/or modules to facilitate removal, this apparatus is configured so that the modules can quickly be removed and replaced without disassembling the modules and without disconnecting or removing the remaining modules from the apparatus. In some embodiments, the apparatus uses quick-release mechanisms for various panels and for the modules themselves to allow the user to quickly remove and replace the modules manually without any tools, or with minimal tools, for example, by use of a single specialized removal tool. This latter aspect may be desirable as it prevents easy removal of the modules by unauthorized personnel and limits module removal to those in possession of the specialized removal tool.

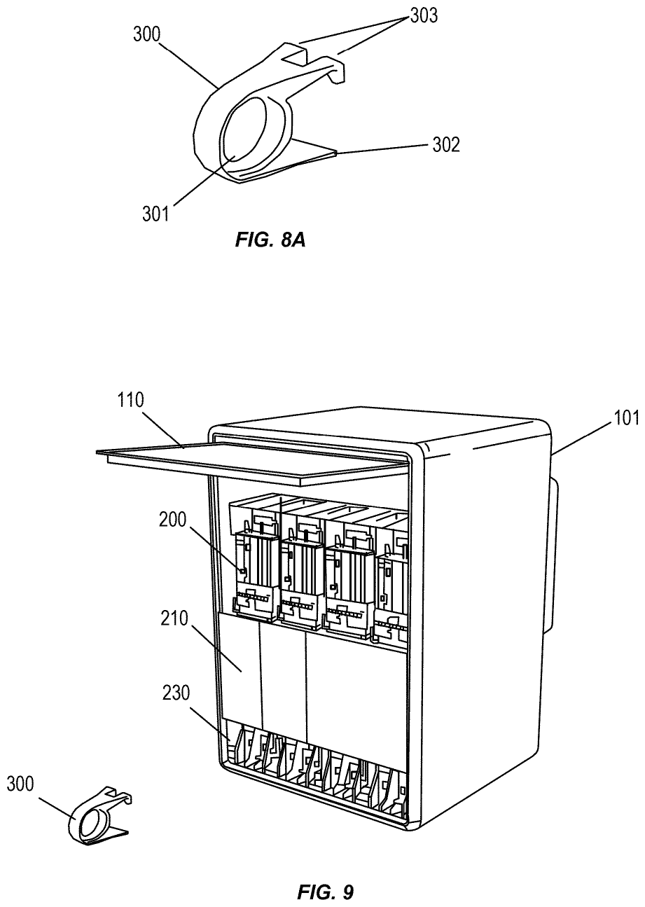

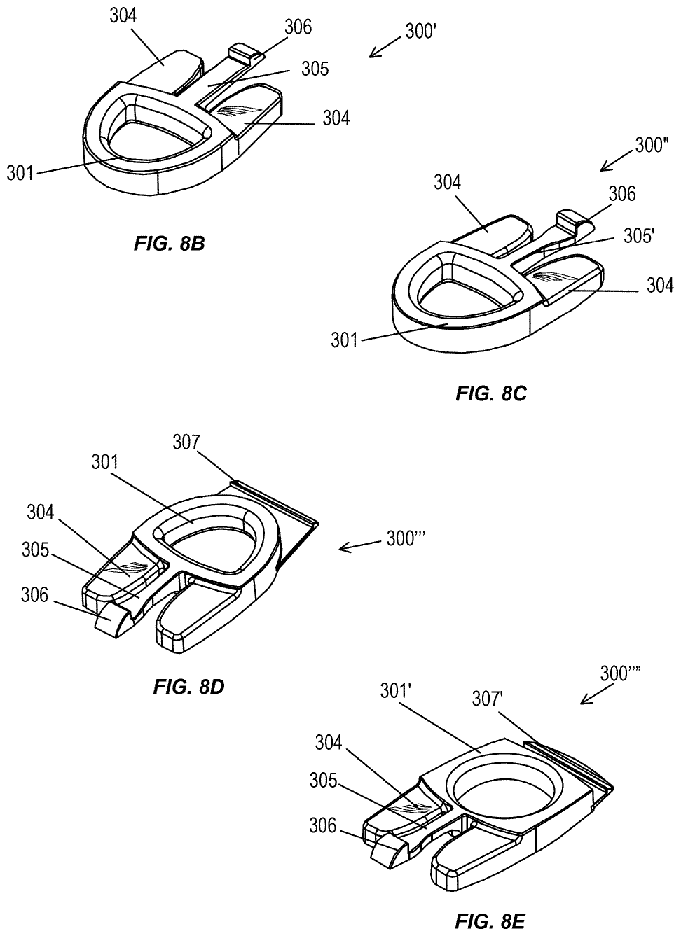

FIG. 8A shows one such specialized removal tool 300 to facilitate removal of a sample processing module from the apparatus enclosure, in accordance with some embodiments. As shown, tool 300 includes a handle 301, a lower tab 302 and an upper pair of hooked tabs 303. In this embodiment, the handle is defined as a finger loop, however, it is appreciated that the handle can be defined according to any shape to facilitate manual use of the tool (e.g. T-shaped tab, flange, etc). Insertion of this tool into a lower portion of the module actuates release of the quick-release mechanism holding the module within the enclosure, while the hooked tabs 303 hook into a corresponding pair of notches in the module so as to allow a user to readily pull the module proximally from the enclosure. The user hooks the tabs 303 over the top edge of the notches in the module, then pushes down so that the lower tab 302 disengages one or more pawls securing the module into place, thereby releasing the module such that pulling by handle 301 removes the module. The rear connectors of the module are configured as plug-in type connectors such that pulling the module proximally also disconnects the module from the power and communication connectors of the apparatus. This removal process is shown in more detail in FIGS. 9-11. FIGS. 8B-8E show alternative designs 300′, 300″, 300′″, 300″″ of the removal tool. In the embodiment of FIG. 8B, tool 300′ has wedges 304 on each side to push in and disengage the pawls of the module when the tool is inserted, and an arm 305 extending to hook 306 that is used to pull the module out. In the embodiment of FIG. 8C, the tool 300″ is similar to that in FIG. 8B except arm 305′ is narrowed which increases the space for the pawls to move in when the wedges 304 are inserted. In the embodiment of FIG. 8D, the tool 300′″ is similar to that in FIG. 8C, except the rearward side of the tool further includes a panel remover tab 307 to facilitate removal of the lower panel that covers the quick-release mechanisms 230 of the module. The user simply flips the tool in the reverse direction, hooks the panel remover tab 307 over the top edge or recess in the lower panel and pulls with the handle 301 to remove the panel. In the embodiment of FIG. 8E, the tool 300″″ is similar to that in FIG. 8C, except the panel puller tab 307′ has been refined to more easily insert the tab in the panel, and the handle 301′ includes a simplified finger hole to facilitate use in both a reverse direction for panel removal and forward direction for module removal.

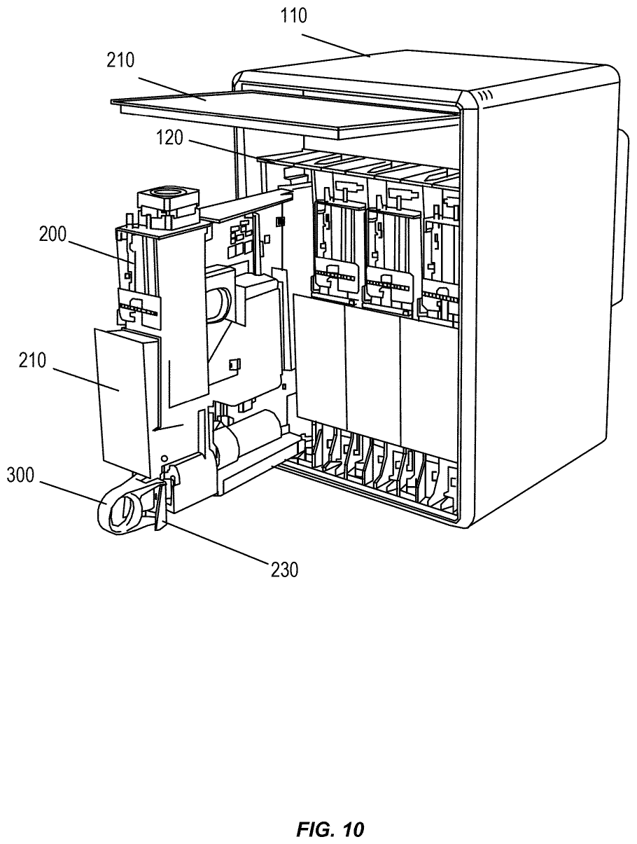

As shown in FIG. 9, the display 110 has been tilted upward, thereby allowing clearance for removal of one or more modules. The upper panel and lower panels have been removed. These panels can also be configured with quick-release mounting (e.g. snap-fit type couplings) such that the panels can be removed manually or with minimal tools. The specialized tool 300 can then be inserted into the quick-release mechanism 230 along the lower portion of the module. As shown in FIG. 10, the tool 300 fits into the lower portion of the module, actuates release of the quick-release mechanism, and hooks into corresponding pair of notches so that the module can be readily removed by pulling the tool 300 proximally.

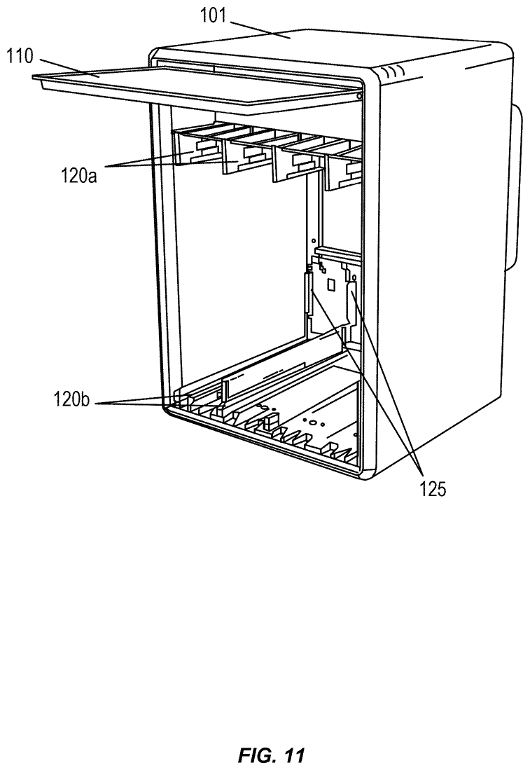

As shown in FIG. 11, each of the modules 200 has been removed in the same manner leaving an empty enclosure. As shown, the enclosure includes tracks, upper tracks 120a and lower tracks 120b for mounting and aligning the modules when placed within. By aligning the modules with the tracks, the modules are precisely aligned such that advancing the modules along the tracks connects the power and communication connections along the rear of the module to the corresponding connectors 125 disposed in the rear of the enclosure as the module is locked into place with the quick-release mechanism 230.

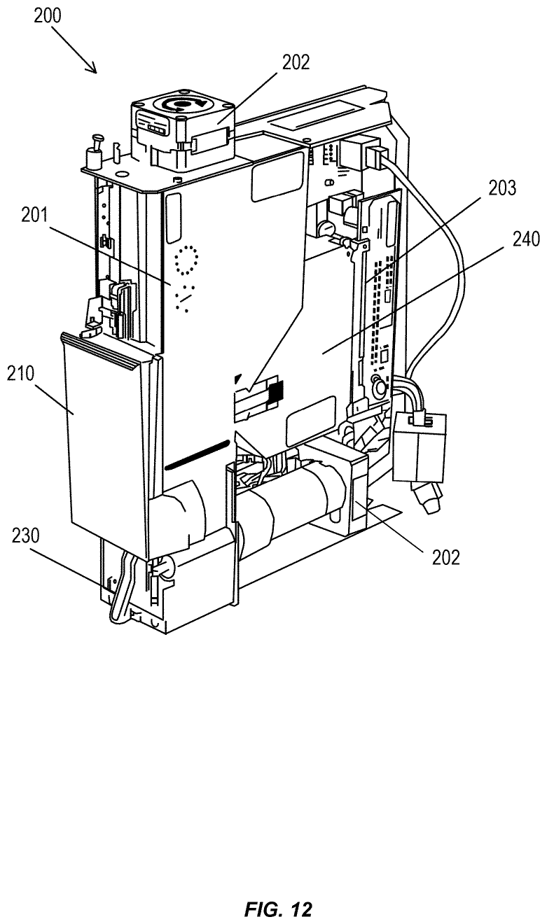

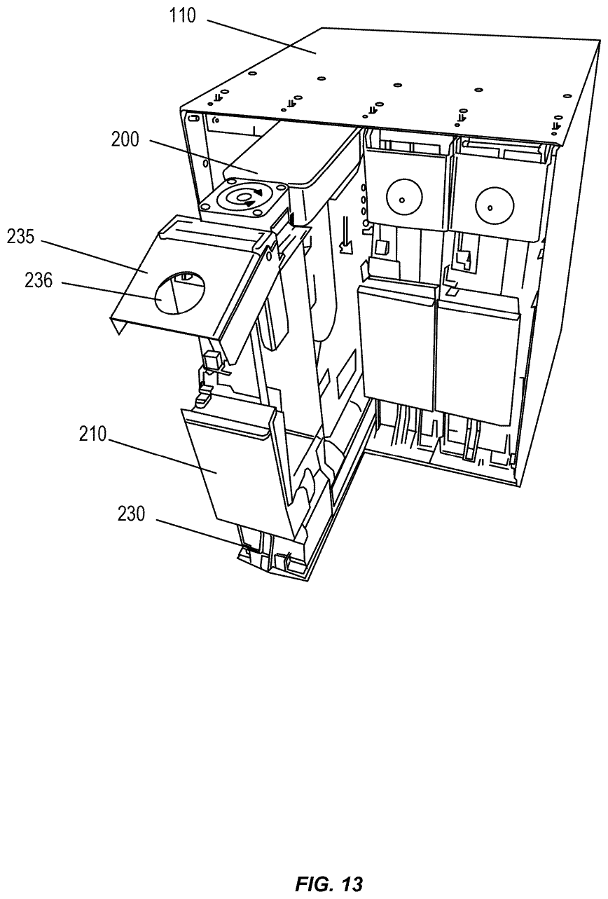

FIG. 12 shows a processing module 200 removed from the apparatus enclosure, in accordance with some embodiments. The module 200 includes a housing 201 that defines an interior receiving bay in which the sample cartridge is received. The module 200 includes a bay door 210 that covers the front opening of the cartridge bay and is movable between an open and closed configuration. The module 200 includes one or more motors 202 to actuate movement of the door, as well as functions of various other components (e.g. valve assembly, syringe assembly and movement of a sonication horn) in order to facilitate processing and analytical testing of a sample within the sample cartridge. The module further includes an instrument assembly 240 that includes a thermal cycling module as well as an excitation/optical detection module to facilitate sample processing and analytical testing. The module 200 further includes a PCB along a rear portion that includes associated circuitry and a microcontroller for controlling the functionality of the various components, and one or more rear facing connectors 203 of a plug-in type so as to connect power and communication with the enclosure upon insertion of the module therein. The lower portion includes quick-release mechanism 230 that actuates between a locked position when slid into a corresponding lower track of the enclosure and an unlocked position when the specialized tool 300 is inserted within.

FIG. 13 shows another processing module 200 being removed from the apparatus enclosure 110, in accordance with some embodiments. This embodiment is substantially similar in construction to the previous embodiment described above, including a bay door 210 and a quick-release mechanism 230 along a lower portion. In this alternative embodiment, the front, upper portion of the module includes a movable release lever or flap 235 with a central opening 236. This feature can also be used to lock and release the module in place. The flap can hinge upward and the central opening 236 of the flap can be used to manually pull the module from the enclosure. After replacement of the module, the flap can be pushed down into a vertical position. In some embodiments, movement of the flap may also engage a quick-release mechanism such that when the flap is vertical the module is locked in place and when the flap is pulled upward the quick-release mechanism is released.

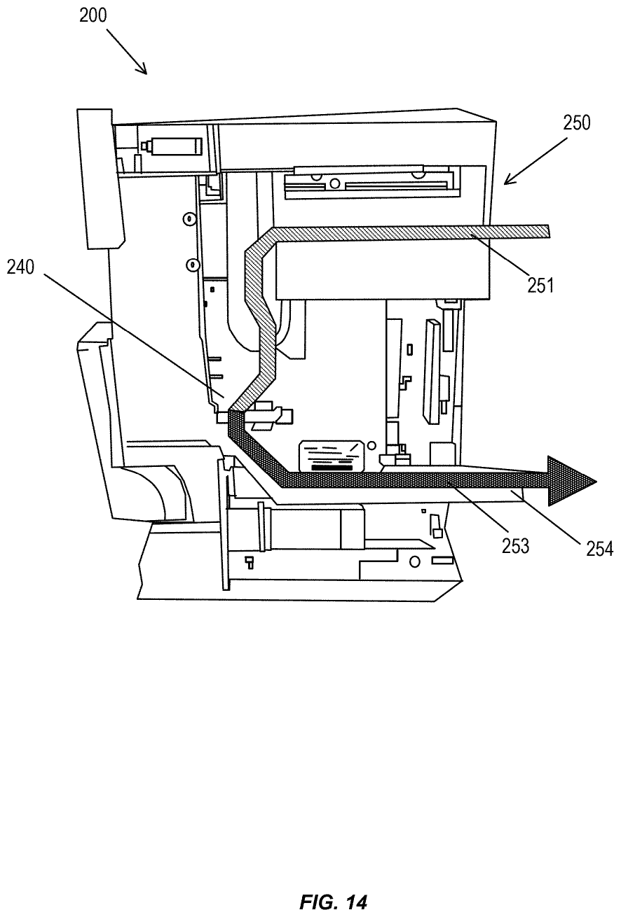

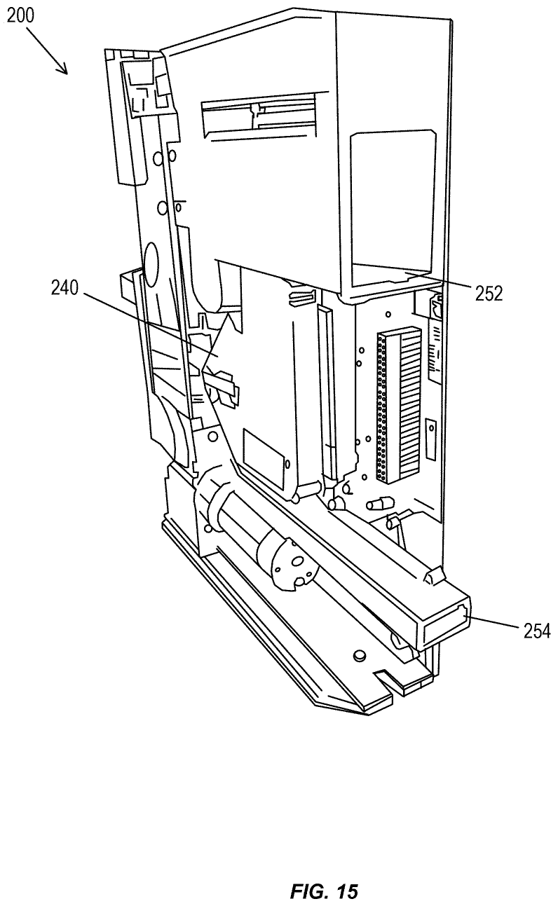

FIGS. 14-15 shows a processing module 200 configured with directed air cooling, in accordance with some embodiments. In a conventional apparatus, the enclosure merely includes a whole-box fan that sucks air into the enclosure and through the modules. While this provides effective cooling, this approach also introduces substantial debris and dirt into the modules, which is of particularly concern when using the apparatus in remote locations (e.g. sub-Saharan regions) where there is an appreciable amount of dust and debris that can foul operation of the device. There are several cooling features that allow for improved cooling within the apparatus enclosure and that avoid the drawbacks noted above. In one aspect, the module has been configured to provide a directed airflow path through the module from an air intake and out through an air outlet or exhaust and avoid introducing air into other locations unnecessarily. In some embodiments, the airflow is defined by one or more portions of the module housing and/or conduits attached to the housing. In another aspect, the power supplies for each of the modules have been relocated outside of the enclosure to reduce the heat generated internally. In such embodiments, the main source of heat generation would then come from the instrument assembly which includes a thermal cycling unit that thermally cycles the sample within a reaction vessel attached to the cartridge.

In the embodiment shown in FIG. 14, the module 200 includes an upper intake 250 along which an air inflow 251 is directed to the instrument assembly 240, which produces heat due to the thermal cycling unit, and then directs the heated air along outflow 253 to exhaust at outlet 254. As can be seen in FIG. 15, the air intake 252 is a large rectangular opening along an upper housing portion of module 200 and the air outlet exhaust 254 is a distal end of a lower conduit positioned below the instrument assembly 240. While a particular cooling path design is shown here, it is appreciated that this directed air concept could be realized in various other ways, for example integrated fully within the module housing or utilizing a lower air intake and upper exhaust.

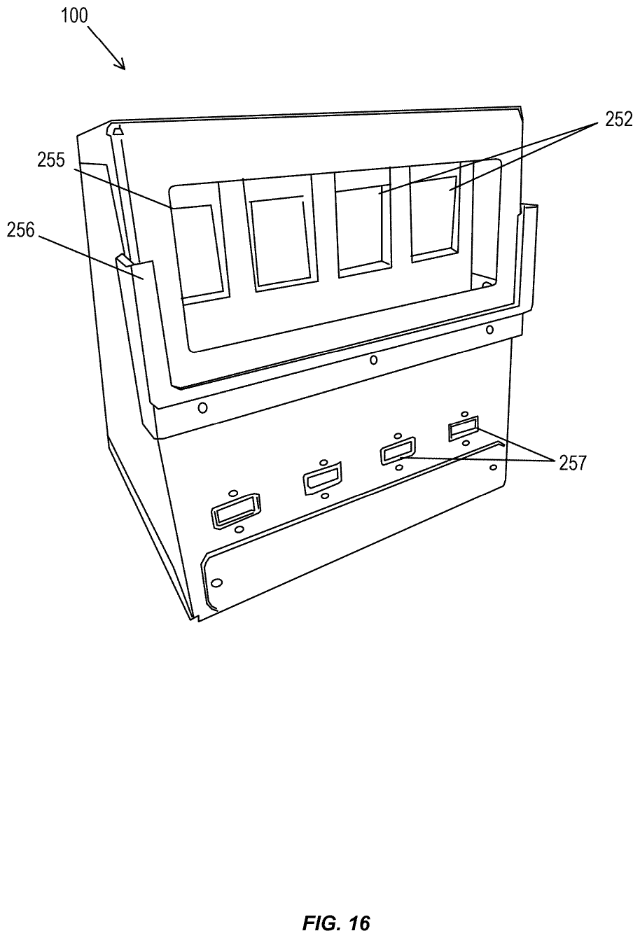

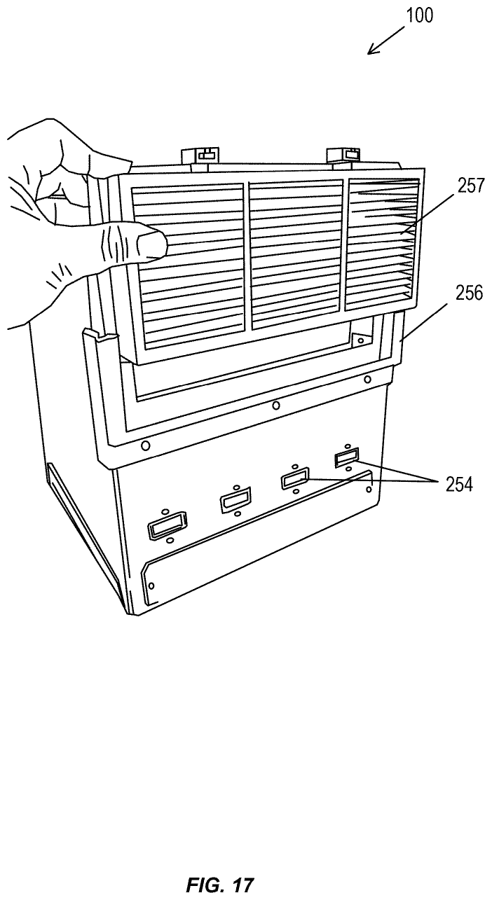

FIGS. 16-17 shows another cooling feature for filtering of directed air cooling, in accordance with some embodiments. In this embodiment, the apparatus 100 includes multiple modules 200 configured with the directed air cooling concept described above. The enclosure housing includes a large upper opening 255 through which the air intakes 252 of the modules are exposed and which includes a filter bracket 256 for supporting a filter. The enclosure housing further includes air outlets 257 that fittingly receive air outlets 254 for exhaust from the modules. As shown in FIG. 17, an air filter 257 can be easily fitted into the filter bracket 256 so as to filter all the air intakes through the modules and prevent introducing dust and debris when cooling the apparatus. This approach allows for easy removal and replacement of the air filter as needed.

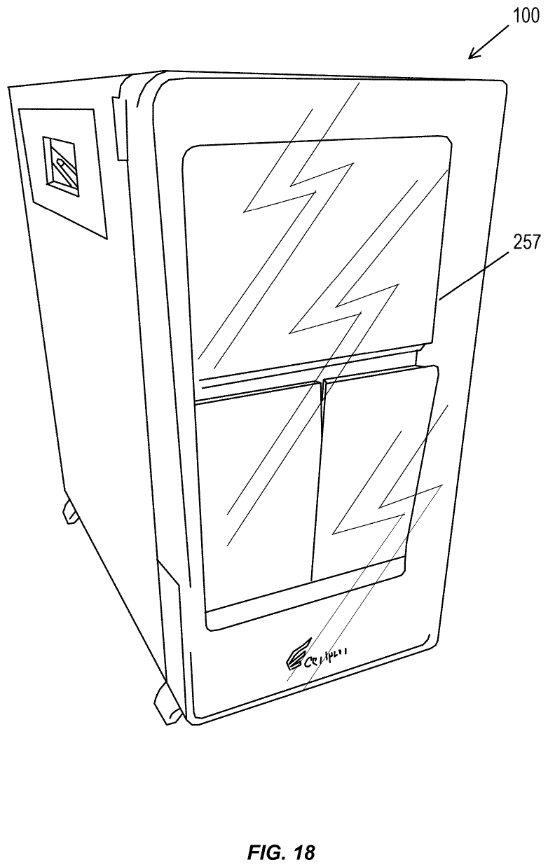

FIG. 18 shows a sealed, closed system in accordance with some embodiments. As noted above, in some embodiments, the apparatus is configured to remove all non-PCR heat sources (e.g. power supplies) from the enclosure. These heat sources can be located outside the enclosure or within a separate enclosure attached thereto. In such embodiments, the only substantial heat sources are the thermal cycling units of the instrument assemblies of the modules, which can be dissipated by thermal conduction while the entire enclosure remains sealed/closed to airflow. This avoids any possible introduction of dust and debris through the airflow. In some embodiments, the enclosure can also include an additional removable barrier 257, which can be a housing, film or cover, that seals the entire enclosure. In some embodiments, the apparatus can utilize the enclosure itself as a heat sink to dissipate heat. The non-PCR heat sources (e.g. power supplies) can be thermally isolated from the enclosure housing so as to avoid contributing additional heat by thermal conduction.

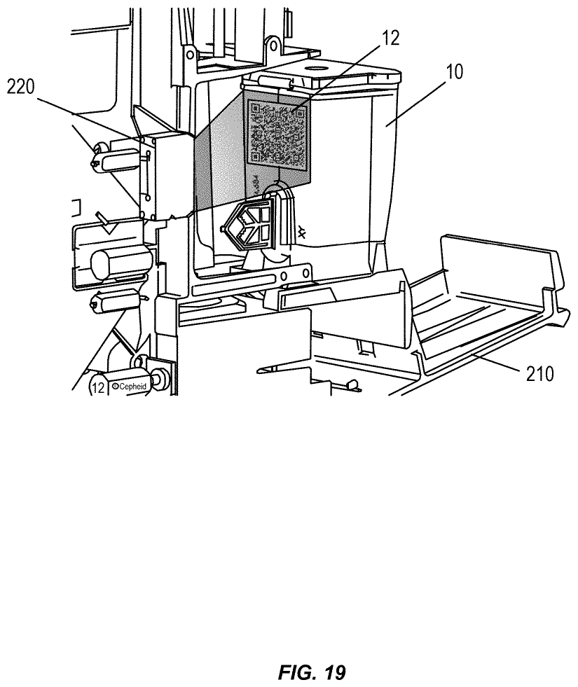

In another aspect, the apparatus can include an improved identifier located within the cartridge receiving bay. The identifier is used to identify any of: a sample cartridge, a sample, and a user badge of personnel. Scanning from within the module bay enables new workflows, and tests to be initiated at the module. Cartridge scanning occurs passively within the module during loading, simplifying workflow and reducing errors. This approach allows an entire test to be performed interacting only with the GX instrument. FIG. 19 shows a sample cartridge being loaded into a sample processing module and being identified by identifier 220 disposed within the receiving bay. The identifier 220 is positioned so that it can scan a barcode 12 on the cartridge 10 as the cartridge 10 is loaded in to the receiving bay.

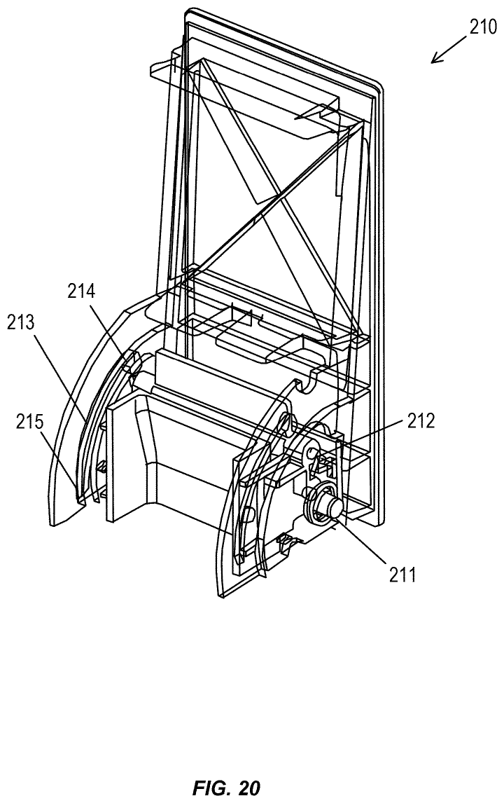

In another aspect, the apparatus can include an improved bay door design, such as that shown in FIG. 20. In this design, a rigid hinge pin 211 provides a smooth and stable pivot. Spring 211 facilitates opening and closing of the door, which includes spring detents that snap door solidly in both standby (e.g., toward a vertical position) and open positions (e.g. toward a horizontal direction). In some embodiments, the door can further include a locked position during running of a diagnostic assay in which the door is pushed slightly inward. In this embodiment, a spring pin (not shown) locks along detents at terminal positions 214, 215 of the groove in curved member 213. These terminal position correspond to the closed and open positions of the door. In this embodiment, the bay door can be pre-hung on a mini chassis/door frame 212. The door can be configured to be compatible with existing earlier generation modules as well. In some embodiments, the door is constructed from sheet metal as a lightly-modified uniframe design.

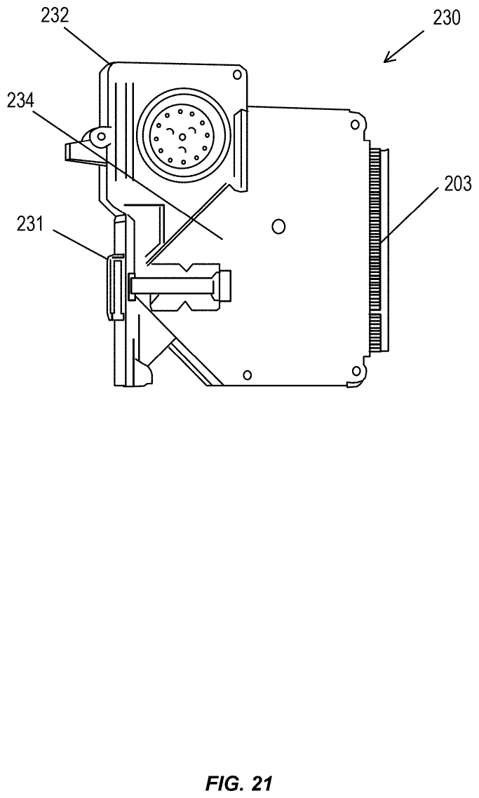

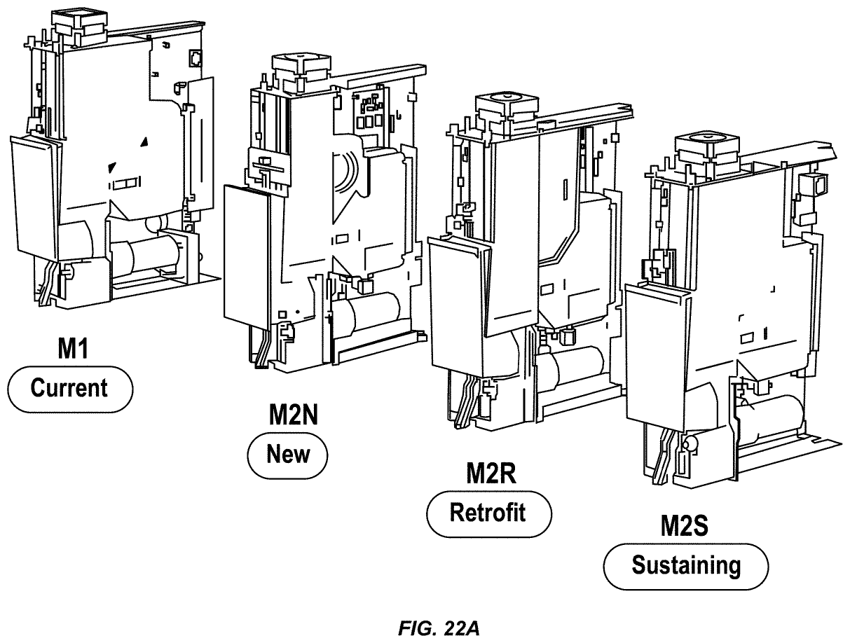

In another aspect, the apparatus can include an improved thermal cycling unit in the instrument assembly 240, such as that shown in FIG. 21. The instrument assembly includes a reaction vessel receptacle 231 for insertion of the reaction vessel containing prepared fluid sample, one or more heaters 232 for thermal cycling the fluid sample, an optical excitation/detection unit 234 for detecting a target analyte in the fluid sample, and the rear facing connector 203. The instrument assembly can further include a fan for cooling the sample. Conventional instrument assemblies of conventional modules utilize two heater plates under single control that heat and cool identically. The improved instrument assembly 230 includes a thermal cycling unit that can be configured for gradient heating/cooling. This is allowed by the use of multiple heaters with individual heater control. In other embodiments, the thermal cycling unit utilizes active cooling by use of thermoelectric coolers (e.g. Peltier devices).

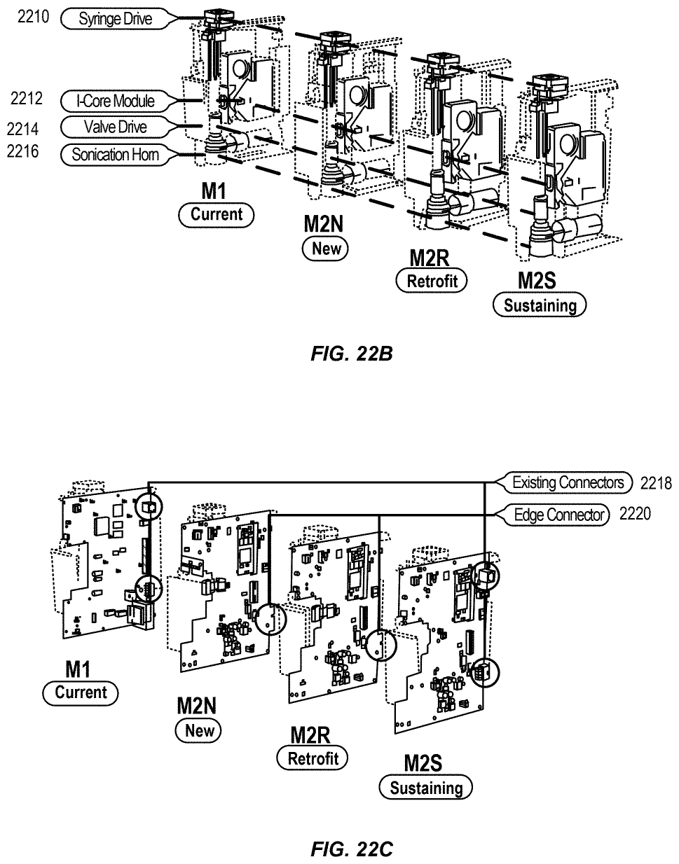

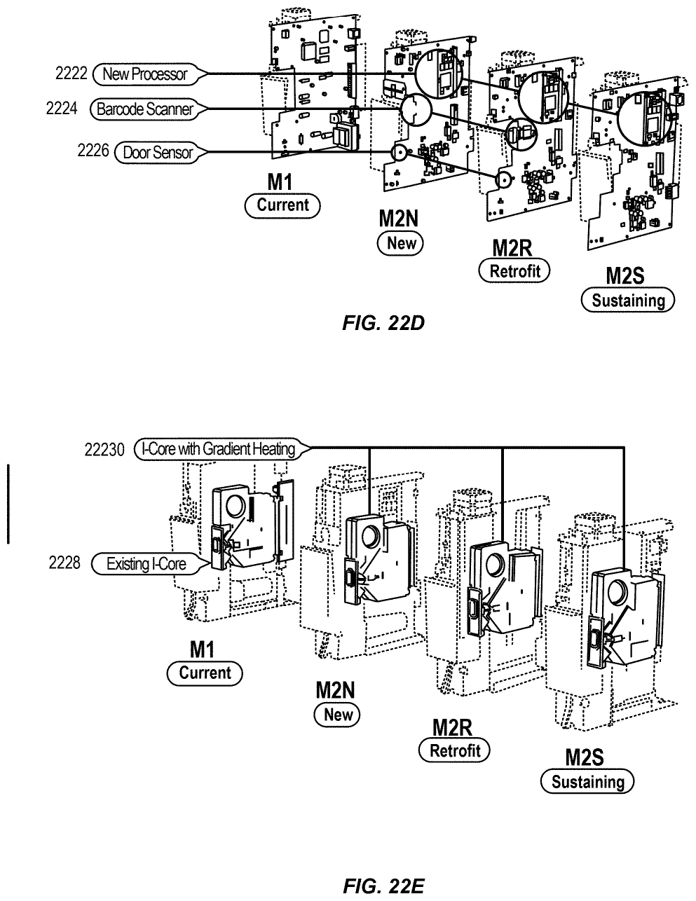

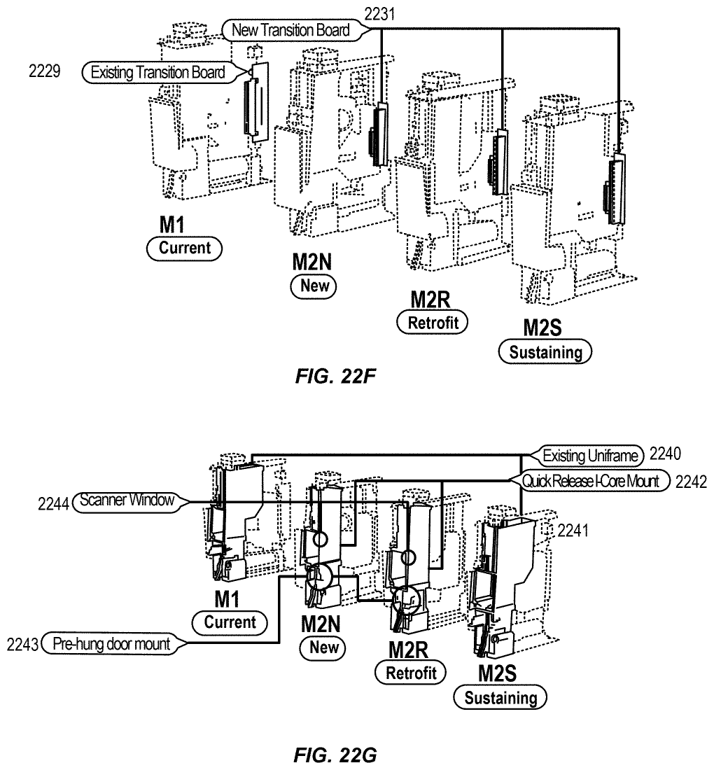

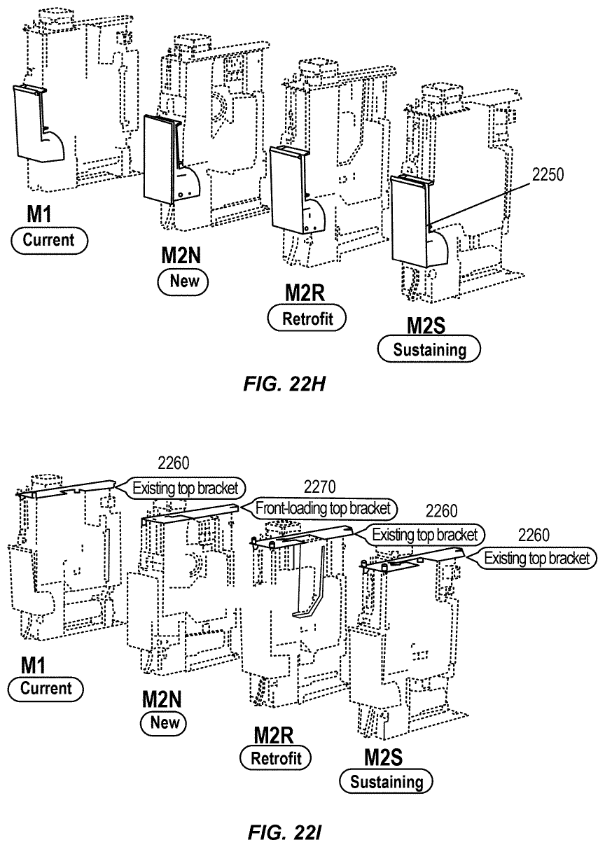

FIG. 22A shows various differing sample processing modules to illustrate variations of replacement modules having backwards and/or forwards compatibility with previous generation apparatuses and next generation apparatuses. For example, module 1 design (M1) refers to a current module design for a conventional apparatus (e.g. GeneXpert). As described above, one problem with such modules is that over time certain components may be designated by the manufacturer as “end-of-life” or may be limited in functionality for next generation products, particularly the motherboard. Therefore, rather than developing an entirely new system and phasing out previous generations, which is the typical standard approach in the industry, it would be advantageous to develop replacement and/or modified modules with new components and/or functionality that remain compatible for use with a conventional apparatus. It would be further advantageous if such modules are of a construction that facilitates updates in newer generations of modules and apparatus. In this manner, the replacement modules extend the useful life of earlier generation apparatus, while facilitating future developments in next generation apparatus.

Examples of such new and/or replacement modules are depicted as Module 2 (M2), and include M2N, M2R and M2S. The M2N module is a “new module” that retains the core technologies of the M1 module for use in the new apparatus described herein. M2N is equipped with front-loading installation and quick-release features so as to be readily removable as described herein. The M2 module replaces “end-of-life” components in the M1 and may include one or more new components with functionality that may or may not be used by the conventional enclosure. The M2R module is designed as a “retrofit module” so as to retrofit the conventional apparatus with new functionality. M2R includes features that fit in non-front loading apparatus. For example, the M2R can include a cartridge identifier (e.g. barcode scanner) within the bay, whereas, the conventional M1 module relied on use of an external peripheral device (e.g. handheld barcode scanner), thereby improving functionality of existing conventional apparatus. M2S is a “sustaining module” that replaces only certain components (e.g. motherboard) that are required to maintain the same functionality as the original M1 module, which allows use of a conventional apparatus to be maintained without adding new functionality. The M2S module is equipped with the same mounts and connectors as the M1 to allow for in field replacement of old M1 modules. This option is advantageous for those users that desire only to extend use of their existing conventional device and operate in the same manner. Thus, M2S is designed as a drop-in replacement of the M1 module with a new motherboard that is used in in all M2 modules, yet still utilize connections and available components that are the same as the M1 module. Any of the M2 modules can be equipped with mountings (e.g. simple brackets along the top and/or bottom) so as to be compatible with earlier generations of apparatus enclosures.

As described, the new M2 modules are designed with certain components and/or modules that are the same, substantially the same, or substantially the same in functionality as the earlier M1 module, while certain other components are different and may be configured to provide the same or similar functionality or entirely new functionality from that of the M1 modules. It is appreciated that some of these updated components may not be utilized by the conventional apparatus of M1, while other components may actually allow updates in functionality of the conventional M1 apparatus.

Examples of designs that maintain certain components while replacing others are shown in FIGS. 22B-221. As shown in FIG. 22B, the syringe drive 2210, instrument assembly module (i.e., I-Core module) 2212, valve drive 2214 and sonication horn 2216 can remain the same across all modules, although it is appreciated that the module could be designed to replace one or more of these components as well. As shown in FIG. 22C, certain aspects of the motherboard remain the same to provide compatibility, for example, the location and type of connectors 2218 and edge connectors 2220. As shown in FIG. 22D, the M2 modules can include certain different components to maintain existing functionality or provide forwards compatibility with new functionality, these components including the processor 2222, integrated barcode scanner 2224 and door sensor 2226. As shown in FIG. 22E, the M2 modules can include an instrument assembly (I-Core module) 22230 that is enhanced with additional functionality (e.g. gradient heating) but still compatible with the connections of the existing instrument assembly module 2228 of the M1 modules. As shown in FIG. 22F, the new instrument assembly module 22230 can include a new transition board 22231 that pulls straight backward to allow the module to be easily installed and replaced, thereby providing forward compatibility. As shown in FIG. 22G, the housing 22241 of the M2 modules may also be modified as compared to the uniframe housing 22240 of the M1 modules. The M2 modules can include a quick-release 2242 mount for easy replacement of the instrument assembly module, a pre-hung door mount 2243, and a scanner window 2244 to facilitate compatibility with an integral cartridge identifier (e.g. barcode scanner). As shown in FIG. 22H, an updated door design 2250 can be designed so as to be compatible with all M2 modules and the M1 modules. The door can include a first mounting component for mounting with the M1 modules and a second mount for mounting with the M2 module. In some embodiments, the second mounting component may have no function when mounted with the M1 module, while the first mounting component may have no function with mounted with the M2 modules. This door design (see FIG. 20) provides improved stability and function as compared to the conventional door design of the original M1 modules. As shown in FIG. 22I, the M2 modules can be equipped with mounts to facilitate mounting within the M1 module enclosure or within the new enclosure described herein. For example, the M2R and M2S modules can be equipped with the same top bracket 2260 as the M1 module while the M2N module can be equipped with a different mount 2280 that facilitates ready insertion and removal from the enclosure described herein to facilitate easier removal and replacement as compared to the conventional apparatus. The M2 module may also include a different airflow path, such as air scoop 2270, to facilitate the improved directed air cooling approach described herein.

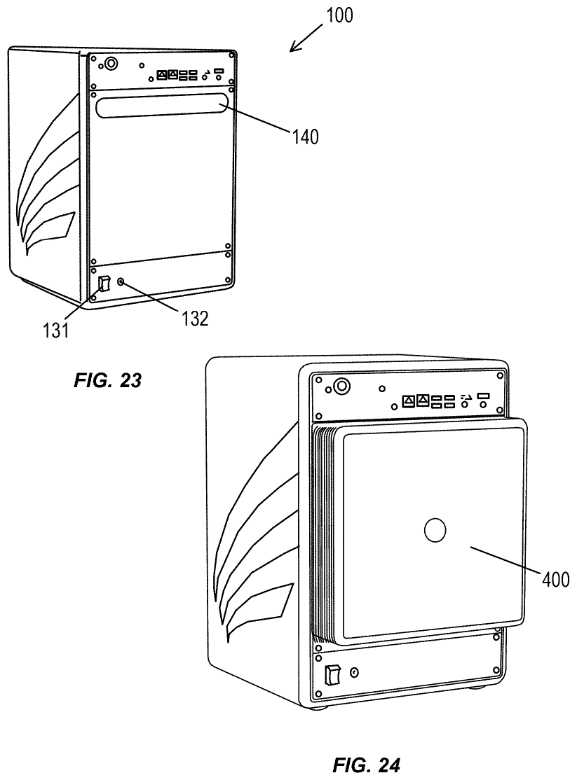

FIGS. 23-34 shows various sample processing apparatus configured for being powered by a direct connection or by a battery pack for portability, in accordance with some embodiments. Typically, in a regular laboratory setting, the apparatus remains at one location and is powered through hard wired connection plugged into power receptacle 132, as shown in FIG. 23. The apparatus is powered on/off by power button 131. In embodiments in which the apparatus 100 operates on external power, e.g. 110V AC, the instrument preferably includes one or more power connections. In some embodiments, power is received though a first connection and output through a second connection. The apparatus 100 can comprise a power supply for supplying power to the instrument and to each module. The power supply may comprise an AC/DC converter for receiving power from an external source and converting it to direct current, e.g., for receiving 110V AC and converting it to 12V DC.

Alternatively, the power supply may comprise a battery. In some embodiments, the apparatus is powered with a specialized portable battery pack, which improves portability for use in remote locations (e.g. sub-Saharan regions) or temporary aid stations. For such embodiments, the apparatus can be equipped with a portable plug-in battery pack 400 that can be plugged into receptacle 140, as shown in FIG. 24. Notably, the portable power pack is optional and the apparatus can be easily switched between a hard-wired power connection and the power pack as needed.

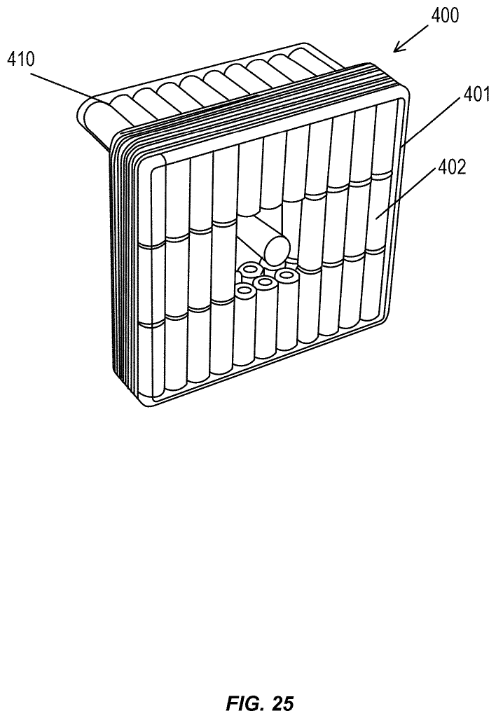

As shown in FIG. 25, the portable battery pack 400 includes a housing 401 that encases multiple rechargeable battery cells 402 and has a plug-in portion 410 that interfaces with receptacle 140 in the rear face of the enclosure housing. In this embodiment, the power battery pack includes 70×21700 rechargeable battery cells that provide 1295 Watt-hours and weighs 10 pounds. This design allows for a battery pack that provides over 9 hours of runtime. Notably, this design locates the power supply for each individual module outside of the enclosure, thereby reducing the heat burden in cooling the interior of the enclosure.

In another aspect, the CPU unit of the apparatus is modular such that it can be easily removed and replaced as needed. As shown in FIG. 26, the CPU unit 150 can be removed from the rear side without disassembling the entire apparatus or removing the modules within. In some embodiments, the CPU unit 150 is the same as that used in the command input module previously described.

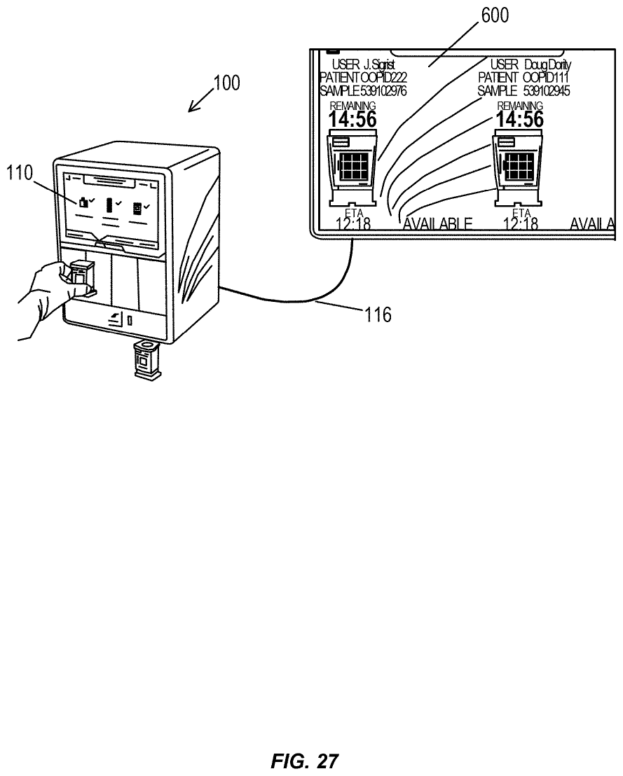

In yet another aspect, the apparatus 100 can include a display output 116 for an external display 600 in addition to the central display, as shown in FIG. 27. This allows the display to be easily viewed by multiple people on-site. The output can be hardwired, as shown, or wireless. In some embodiments, the display output displays the same data as central display 110, while in other embodiments, the display can be configured to show other information, such as a status information for one or more modules. For example, the display output may display a status indicator of all modules, while a user loads one module and views information pertaining only to that module on the central display 110.

Remote Monitoring of Assay Status

In yet another aspect, the system can include a feature that allows a user to monitor the status and/or a result of an assay remotely. Such a feature is particularly advantageous given the nature of conducting assays with the respective sample processing modules, which often take half an hour or more. Many conventional diagnostic systems utilize a single-use, consumable test cartridge that is processed by a non-portable instrument on a laboratory bench top or floor. A user of the system is required to load cartridges into the instrument, and view status, alerts and results through the graphical user interface displayed on the instrument-mounted touchscreen of the instrument. As a test typically takes half an hour or more to run and show results, a user may walk away from the processor to attend to other matters, returning when they estimate the test will be done. Some users set one or more alarms, which can be become cumbersome, particularly when the user is running multiple differing assays concurrently. This remote monitoring feature allows the user to untether from the systems processor and receive continuous real-time information on the test(s) being run without having to estimate completion times or use multiple alarms. The instrument sends real-time test status and results to a server, which provides the information on a website accessible remotely by the user. In some embodiments, the instrument updates the information being displayed in response to any change in the status of the assay being performed (e.g. completion, error, stoppage, result, etc.). It is noted that the test results are not stored indefinitely on the server utilized in remote monitoring. In some embodiments, after a set period of time (e.g. hours, days, weeks) and/or confirmation by the user, the status and result information is no longer available or stored on the server associated with the remote monitoring website. The exact period of time can be set by a system administrator. This further enhances the security of the test result. The full test result can be stored longer or permanently in other systems/servers for access by the physician or the patient's electronic medical record. Additionally, in some embodiments, the testing apparatus only stores the test results for a pre-set period of time (e.g. hours, days), typically two days, which further reduces the possibility of test result information being improperly accessed or disseminated.

In some embodiments, this feature is achieved by use of a URL that is updated with status information by the system so that a user can view the URL from any internet-ready device. In some embodiments, this entails the user scanning a unique QR code generated by the system and displayed on the user interface by the user's mobile device (e.g. smartphone, tablet). The QR code directs the user's device to the URL, so that the user can continue to monitor the status of the assay from a remote location. In other embodiments, the system texts or emails the user a link to the URL so that the user can monitor the status of the assay from any internet-ready device (e.g. desktop device, tablet, smartphone).

In some embodiments, the running test screen and test result screen on the instrument have a QR code. The QR code contains a link (URL) to the server. To provide a basic level of security, the link contains a hash generated from the instrument ID and test ID which is used to look up the test information on the server. It is at least 256 bits and preferably more bits long. For example if the link uses A-Z,a-z,0-9×16 characters the result is 992 bits which results in 4.18×10⁹⁸ possible combinations. Since the chance of finding any test let alone a specific test by searching the links would be infeasible, this approach provides a sufficient level of security for the information being displayed despite being accessible via the internet. In some embodiments, the server could increase this time further by limiting the number of searches from sources of too many searches to one per second or one per minute. In some embodiments, the link could also be an app URL that opens the test information in a custom test viewer app. In this manner, the system can allow for display of advanced data that may include more sensitive information, such as the patient ID or name, the test result, and technical details of the test. It is appreciated that various other approaches to authentication of the user can be utilized in order to provide more advanced data sets having more sensitive patient information.

In one aspect, the above noted features can be used for each of multiple assays being performed concurrently by multiple modules. The user can select the desired notification option and/or scan the unique QR code associated with each assay so as to show the status and/or test result on their device (e.g. mobile device). By providing a unique link/website for each assay being performed, the user can open each on a unique tab in their native web browser of their internet-ready device (e.g. smartphone, tablet), so that multiple tests can be monitored by multiple tabs in their browser. In some embodiments, the user can forward the link in an SMS to other users or can display the QR code on their device for scanning by other users to allow remote monitoring by multiple people.

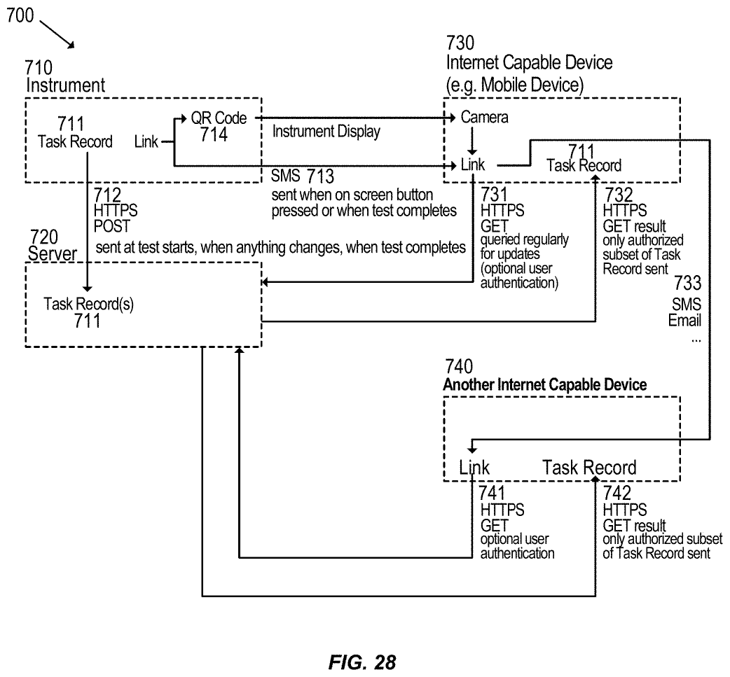

FIG. 28 shows a flowchart 700 illustrating a system configuration by which the instrument, server and user devices (e.g. internet capable device) communicate to facilitate remote assay monitoring in accordance with some embodiments. The instrument 700 generates a task record 711 and sends this as an HTTPS output 712 to server 720. In some embodiments, the instrument sends the output when the assay test starts, when any change occurs (e.g. error, stop), and/or when the assay test completes. The instrument 700 also generates a link at which the task record can be accessed by one or more internet ready devices. In some embodiments, the instrument sends the link as an SMS 713. This SMS message can be sent automatically based on pre-sets or upon selection by a user. In some embodiments, the instrument generates a QR code 714 that is displayed on an instrument display and can be detected by the user's device, typically mobile device (e.g. smartphone, tablet), which directs the user's device to the website link. In this embodiment, the user's device send an HTTP GET request 713 for a status updates on the assay test, and in response, the server sends an HTTPS GET result 732 of the task record 711. In this embodiment, the task record 711 can include differing levels of information, such as a basic data set or an advanced data set. The server can send the basic data set to any user requesting the status update and can send the advanced data set with additional sensitive data regarding the assay to users providing verified authentication. Typically, the basic data set includes only basic information regarding the test assay (e.g. task ID, time parameters, user ID, instrument or module ID, cartridge information, etc), while the advanced data set can include more sensitive patient specific information (e.g. patient ID or name, test ID or type, sample result). By this communication scheme, the user can continue monitoring of the status of the assay remotely through their device and return to the instrument as needed, or after the task record indicates that the test is complete. Additionally, the user can direct other personal to take actions depending on the task record response (e.g. error message, termination, test completion), or can notify others of the test result based on the advanced data set of the task record. In some embodiments, the user may share monitoring of the assay with other users, for example, other team members or personnel. For this aspect, the user may simply send the link (received by SMS or through the QR code) as an SMS or email message 733 to another internet capable device 740, which may be another device associated with another user. Similar to the previous communications described, the other user can then follow the link, which sends an HTTPS GET request 713 to the server 720 and in response, receives an HTTPS GET result 742 of the task record with the data set corresponding to the authorization of the user (e.g. basic or advanced).

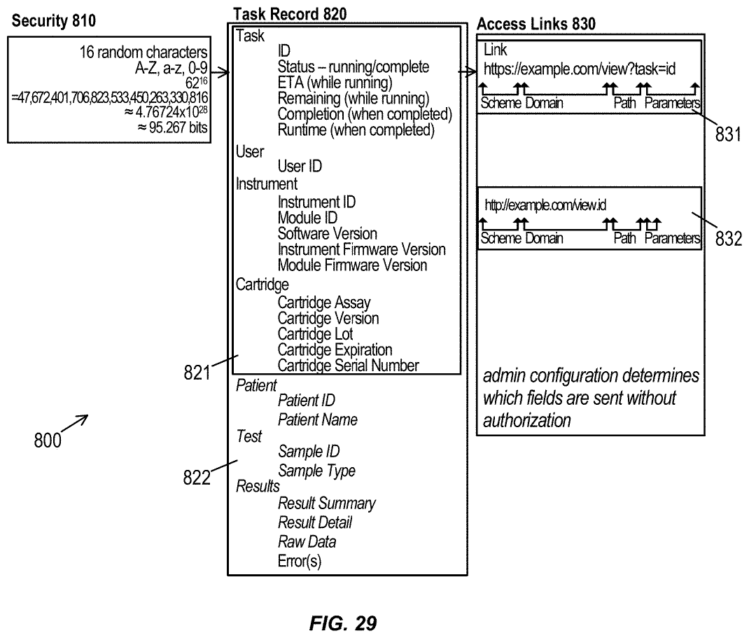

FIG. 29. shows a schematic 800 by which the instrument generates secure access links by which the task record information can be accessed by a user for remote monitoring. While a certain sequence is shown, it is appreciated that these could be performed in any order or could include additional steps.

In a first aspect, the system establishes a basic security 810 scheme for the task information as an obstacle to unauthorized access. In this embodiment, the system generates any suitable number of random characters (e.g. 16 characters, 24 characters, etc) for use in the online accessible http link address. For example, it is estimated that, by generating 16 random characters for use in the link, at one million attempts per second, it would take 15 trillion years to search all possible combinations of 16 characters. If more security is desired, larger sets of random numbers can be used, or alternative security means could be utilized. Moreover, even if a party were to somehow access the http link, only a basic data set 821 would be accessed, which still does not identify the patient or test result, such that the privacy of the patient's health information is maintained. Next, the task record 820 of task information (or a subset thereof) that is accessed remotely is defined. In this embodiment, the task record information includes a basic data set 821, which includes basic task information (e.g. ID, status, ETA, time attributes), user information (e.g. user ID, type, classification), instrument information (e.g. ID, module, software or firmware versions for instrument or module), and cartridge information (e.g. assay, version, lot expiration, serial number). In some embodiments, the task record 820 can further include an advanced data set 822 that includes additional data, which can include patient information (e.g. ID, name, location), test information (e.g., ID, type), and result information (e.g. summary, detail, raw data, error data). It is appreciated that each of the basic data set and advanced data sets could omit one or more of fields of this data or include one or more various other fields of data. In some embodiments, the advanced data set requires additional authorization by a user accessing the link (e.g. password protected, higher security protocol, etc.). Any suitable authorization scheme or specialized application could be used for this feature. The admin configuration determines which fields of data are sent without authorization. Next, the task record is sent securely to the server via HTTPS POST at the online-accessible access link 830 (e.g. https://example.com/save?field=value&field=value . . . ), which is also sent to the user by SMS or email or associated with the QR code. The https can securely transmit the path and the parameters and returns the task record result (see link 831), or the parameters can also be sent as a part of the path (see link 832).

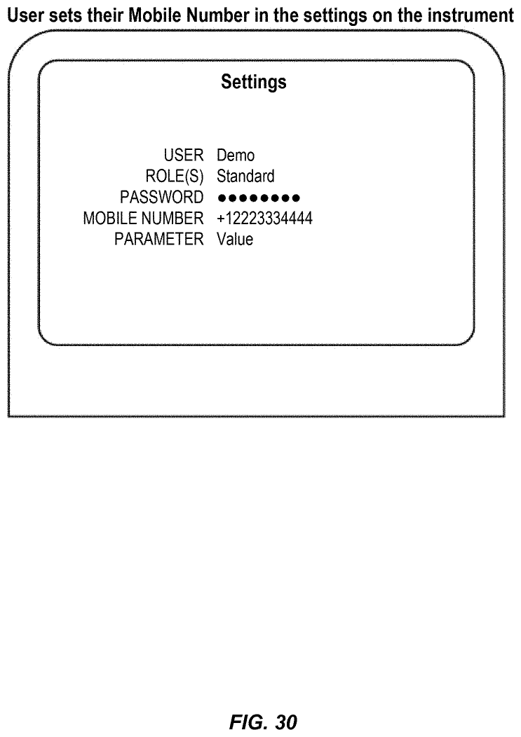

FIG. 30 shows a screen shown on the instrument display by which the user sets their mobile number. It is noted that this setting is associated with a particular user such that when the user is logged on (e.g. by scanning user's badge) and conducting an assay in one or more modules, upon selection of the notification options, the instrument will send the access link for remote monitoring by SMS to the user's number stored in this setting.

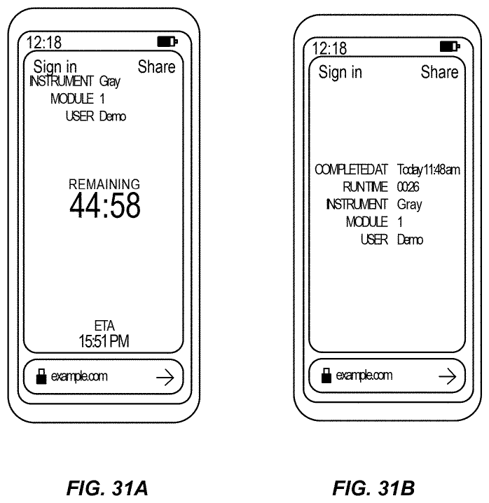

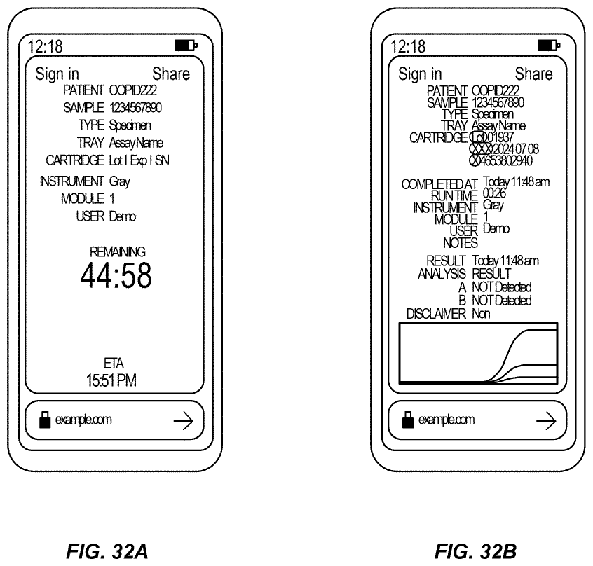

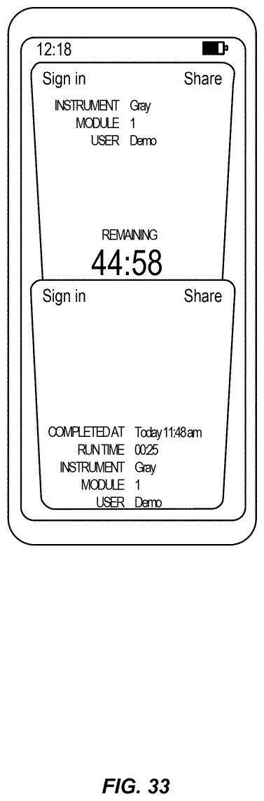

FIGS. 31A-31B and 32A-32B depict example screens that may be displayed on the user's mobile device when using this remote monitoring feature. It is appreciated that remote monitoring may pertain to various differing operations or events, including but not limited to: status, elapsed time, errors and results of the assay. Further, some users may only be interested in the basic data set, while other users may be interested in accessing an advanced data set upon user authentication. In some embodiments, a user may select an option to remotely monitor each or any of these differing operations or events. For example, for basic data sets, some users may want a constantly updated display of the elapsed time, as shown in FIG. 31A, while other users may only want to be notified when the test completes, as shown in FIG. 31B. For the advanced data set displays, when authorized, the running elapsed time display may show additional detailed information regarding the patient and sample in addition to the running elapsed time, as shown in FIG. 32A, and the test result display may show the actual test result, and optionally technical data (e.g. fluorescence detection graph), as shown in FIG. 32B. When a user is monitoring several assays being run concurrently, the user can receive multiple access links from the system, which can be opened in differing tabs, such as shown in FIG. 33, so that the user can scroll through all pending assays and monitor each accordingly.

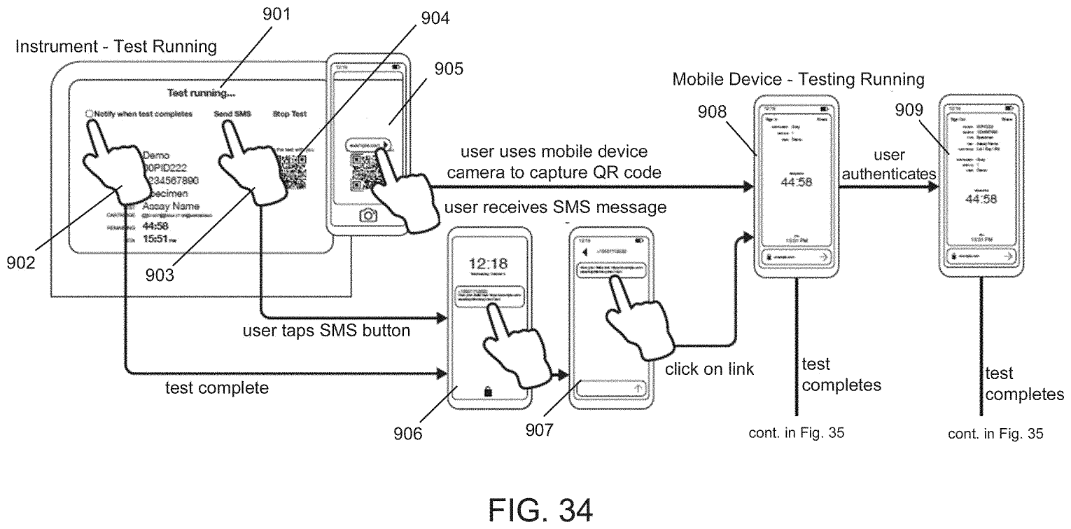

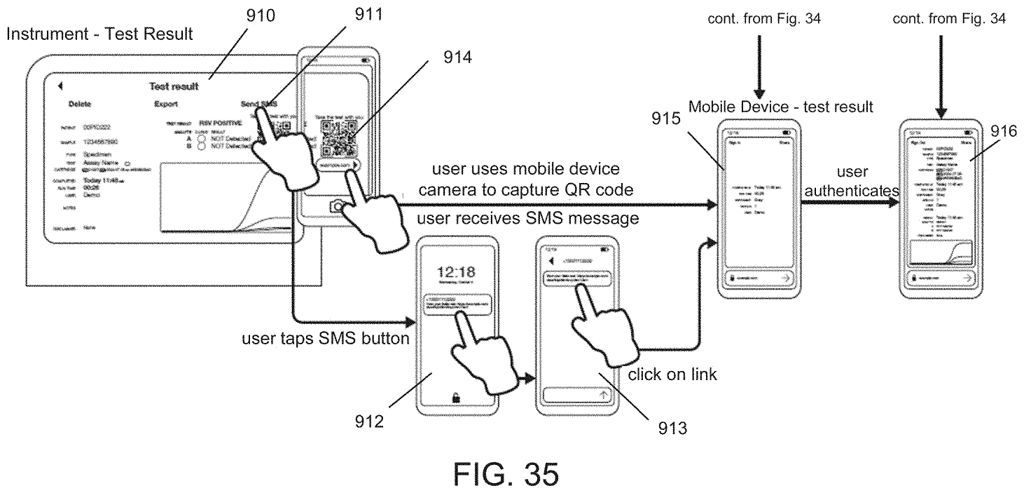

FIGS. 34-36 show flowcharts of a sequence of operations for remote assay monitoring schemes, as described herein. It is appreciated that these flowcharts are exemplary of the remote monitoring concepts described herein and could include variations and alternative options or additional steps and remain in keeping with the inventive concepts described herein.

FIG. 34 pertains to a remote monitoring of running of an assay test. After the user has logged into the instrument and started an assay in a module, the graphical user interface display 901 of the instrument indicates that the test is running and displays task information, as well as various options for selection by the user, including: (option a) notify when test completes; (option b) send SMS and (option c) stop test. The display further shows a QR code 904. If the user selects “option a”, when the test completes, the instrument outputs a communication (e.g. SMS) to the user's internet-ready device (e.g. mobile device) 906 that includes an access link, that when selected 907, directs the user's device to the online website that display the assay status (e.g. test complete/completion time). If the user selects option b, then the instrument immediately outputs a communication (e.g. SMS) to the user's internet-ready device (e.g. mobile device) 906 that includes an access link, that when selected 907, directs the user to the online website showing a real-time display of the current status of the assay test (e.g. pending, elapsed time, completion time). If the user prefers to utilize the QR code for remote monitoring, the user holds their mobile device so as to scan or capture the QR code 904 then the user scans the QR code with their device 905, which then displays the access link, that when selected, directs the user to the website that display the real-time status of the assay test 908 (e.g. pending, elapsed time, completion time). In any of these approaches, the system can be configured to allow for an additional authorization by any suitable means (e.g. password protection, specialized app, user authentication, device authentication) to return an advanced data set with additional identifying 909 of pertaining to the assay.

FIG. 35 pertains to a remote monitoring of running of an assay test result. After the user has logged into the instrument and started an assay in a module, the graphical user interface display 910 displays various test result options for selection by the user, which can be displayed and selected before, during or after performing the test. These options can include: delete, export, and send SMS. If the user wishes to remotely monitor the rest result, the user can select “Send SMS” 901, which sends a immediately sends a communication (e.g. SMS) to the user's internet ready device 912 displaying the access link, that when selected/clicked by the user 913 directs the user's device to the online accessible website 915 displaying the basic data set as to the test result 915. Alternatively, if the user wishes to utilize the QR code displayed on the instrument, the user can capture the QR code with their mobile device 914, which returns the access link that when selected/clicked by the user directs the device to the online accessible website displaying the basic data set of test result 915. In either approach, upon further authentication, the system can return an advanced data set with additional information 916 of the assay test result, including patient information, test information and/or technical details regarding the result.

Although the above description contains many specificities, these should not be construed as limitations on the scope of the invention, but merely as illustrations of some of the presently preferred embodiments. Many possible variations and modifications to the invention will be apparent to one skilled in the art upon consideration of this disclosure.

1. A biological sample processing apparatus comprising:
an enclosure comprising a housing with a front opening;
a plurality of sample processing modules held within the enclosure, each sample processing module configured to hold a removable sample cartridge and perform sample processing on a sample within the corresponding removable sample cartridge,
wherein each module is independently operable; and
wherein each module is readily removable from the enclosure and replaceable without disassembly of the module or enclosure.

2. The sample processing apparatus of claim 1, wherein each of the modules includes at least: a valve drive, a syringe drive, a sonication horn and an instrument assembly.

3. The sample processing apparatus of claim 2, wherein the instrument assembly includes a thermal cycling module and an optical excitation/detection module, wherein the instrument assembly is removable as a module.

4. The sample processing apparatus of claim 1, wherein each of the modules includes a plurality of connectors thereon that couples with a corresponding plurality of connectors within the enclosure when held within the enclosure.

5. The sample processing apparatus of claim 1, wherein the plurality of sample processing modules comprises between 2 or more modules.

6. The sample processing apparatus of claim 1, wherein the plurality of sample processing modules comprises between 2 and 100 modules.

7. The sample processing apparatus of claim 5, wherein the plurality of sample processing comprises between 2 and 16 modules.

8. The sample processing apparatus of claim 1, further comprising:
one or more removable panels that partly cover the front opening of the enclosure housing.

9. The sample processing apparatus of claim 1, further comprising:
a central display that displays status information as to one or more of the modules.

10. The sample processing apparatus of claim 9, wherein the central display is a touch display and configured to selectively display status information as to any of the one or more modules therein upon receiving a selection by a user via the touch display or automatically.

11. The sample processing apparatus of claim 9, wherein the central display is tiltable to one or more positions to facilitate improved interaction by the user during use.

12. The sample processing apparatus of claim 9, further comprising:
a display output that outputs a display signal to an external display in addition to that displayed by the central display.

13. The sample processing apparatus of claim 1, wherein the enclosure and associated plurality of connectors for each module are configured for compatibility with a plurality of differing types of modules.

14. The sample processing apparatus of claim 13, wherein the plurality of differing modules include earlier generation modules having one or more discontinued components and next generation modules having one or more updated components.

15. The sample processing apparatus of claim 14, wherein the earlier generation modules and next generation modules are functionally identical in regard to any or all of: a valve drive, a syringe drive, a sonication horn and an instrument assembly, or any combination thereof.

16. The sample processing apparatus of claim 14, wherein the earlier generation modules and next generation modules differ functionally in regard to any or all of: CPU connectivity, a communication module, module servicing, a cooling system, a sample cartridge identifier, a door mechanism, a thermocycling unit or any combination thereof

17. The sample processing apparatus of claim 1, wherein each of the modules are held within the enclosure by one or more quick-release mechanisms.

18. The sample processing apparatus of claim 17, wherein each of the plurality of modules is configured with a plurality of connectors disposed along a rear side thereof and one or more retraction features on a front side thereof to facilitate removal and/or insertion of the respective module.

19. The sample processing apparatus of claim 18, wherein the retraction feature comprises one or more notches or opening for receiving a tool or finger for removal of the module from within the enclosure.

20. The sample processing apparatus of claim 17, wherein each of the plurality of modules is configured to be slidably received within the enclosure from a front side thereof.

21. The sample processing apparatus of claim 20, wherein each of the modules includes upper and lower brackets configured to interface with corresponding upper and lower tracks in the enclosure.

22. The sample processing apparatus of claim 1, wherein each of the plurality of modules is configured to be removed and/or replaced manually without any tool.

23. The sample processing apparatus of claim 1, wherein each of the plurality of modules is configured with a fingerhole for manually removing the module without any tools.

24. The sample processing apparatus of claim 1, wherein each of the plurality of modules is configured to be removed and/or replaced manually with a single tool.

25. The sample processing apparatus of claim 24, wherein the single tool comprises a handle and one or more tabs for actuating a quick-release mechanism and engaging the module to allow removal of the module by pulling on the handle.

26. The sample processing apparatus of claim 1, wherein each module comprises an air intake and an air outlet to direct an airstream through the module for cooling.

27. The sample processing apparatus of claim 26, wherein the air intake and air outlet are disposed on a rear side of the module and configured to align with a corresponding air intake and air outlet of the enclosure.

28. The sample processing apparatus of claim 27, wherein one or both of the air intake and air outlet of the enclosure comprises one or more openings.

29. The sample processing apparatus of claim 27, wherein the enclosure further comprises one or more filters that cover the air intake of the enclosure.

30. The sample processing apparatus of claim 1, wherein the entire enclosure is sealed from airflow therein by a secondary housing, film or cover.

31. The sample processing apparatus of claim 30, wherein the apparatus is configured such that a power supply for each of the modules is disposed outside of the housing of the respective module.

32. The sample processing apparatus of claim 31, wherein the enclosure is configured so as to be thermally coupled to an interior of the enclosure to cool by acting as a heat sink.

33. The sample processing apparatus of claim 32, wherein the power supplies are thermally insulated from the enclosure.

34. The sample processing apparatus of claim 1, further comprising:
a single removable battery pack for electrically coupling with the power receptacle for powering the plurality of modules.

35. The sample processing apparatus of claim 34, wherein the battery pack is configured to releasably couple to a rear connector on the enclosure while a majority of the battery pack remains outside of the enclosure.

36. The sample processing apparatus of claim 34, wherein the power pack comprises a plurality of rechargeable battery cells.

37. The sample processing apparatus of claim 1, wherein the apparatus comprises:
a central display; and
a display output configured to communicatively couple with an external screen.

38. The sample processing apparatus of claim 1, wherein the apparatus is configured to output to the external screen a status indicator or instructions for each of the modules individually or in combination.

39. The sample processing apparatus of claim 1, wherein the external screen is any of: an external monitor or display, a smartphone screen and a tablet screen.

40-79. (canceled)
